(19) United States
(12) Patent Application Publication
(10) Pub. No.: US 2023/0062579 A1
(43) Pub. Date: Mar. 2, 2023

(54) ACTIVITY-SPECIFIC CELL ENRICHMENT

(71) Applicant: ABSCI Corporation, Vancouver, WA (US)

(72) Inventor: Jia Liu, Vancouver, WA (US)

(21) Appl. No.: 17/793,171

(22) PCT Filed: Jan. 15, 2021
(86) PCT No.: PCT/US2021/013734

§ 371 Date: Jul. 15, 2022

Related U.S. Application Data
(60) Provisional application No. 62/961,392, filed on Jan. 15, 2020.

(51) Int. Cl. C12N 15/65; C12N 15/70

(52) U.S. Cl. CPC C12N 15/65; C12N 15/70

(57) ABSTRACT

An activity-specific cell-enrichment method, capable of selection of high-performing host cells and/or expression vectors from a genetically diverse pool of host cells that can comprise expression vectors is provided.

### CROSS REFERENCE TO RELATED APPLICATION

This application claims the benefit of U.S. Provisional Patent Application No. 62/961,392, filed Jan. 15, 2020, which is incorporated herein by reference in its entirety.

### FIELD

The present disclosure is in the general technical fields of molecular biology and biotechnological manufacturing. More particularly, the present disclosure is in the technical field of host cell engineering for gene product expression.

### BACKGROUND

Production of biotechnological substances is a complex process, subject to multiple factors that affect the quality and quantity of gene products, such as proteins, expressed by host cells. Given a population of host cells comprising expression constructs, where there is variation (diversity) among host cell genomes and/or expression constructs, it would be advantageous to select from that diverse population the host cells and/or expression constructs capable of producing the desired amount of active gene product per cell. The technical challenges to accomplishing this are more difficult to overcome when the gene product is expressed entirely in the host cell cytoplasm, and thus cannot easily be contacted by gene-product-specific detection reagents.

### SUMMARY

There is clearly a need for improved methods for selecting high-performing host cells and/or expression constructs. The present disclosure provides methods for activity-specific enrichment of high-performing cells from a genetically diverse population of host cells that can comprise expression constructs.

Thus, in some embodiments, methods for selecting expressing host cells from a population of host cells having a genetic diversity, the genetic diversity comprising a plurality of genetic variants, wherein at least some of the host cells comprise a polynucleotide sequence encoding a gene product of interest are provided. In some examples, the method includes culturing the population of host cells, whereby the gene product of interest is expressed by a subpopulation of the host cells of the population, the subpopulation thereby comprising expressing host cells, wherein levels of the expression of the gene product of interest from the expressing host cells varies based on the genetic variant; labeling at least some of the expressing host cells of the subpopulation, wherein the labeling comprises associating the gene product of interest with a detectable moiety, wherein an amount of the labeling is proportional to the expression level of the gene product of interest in the expressing host cell, thereby producing labeled expressing host cells; and selecting a subset of labeled expressing host cells, wherein the selecting comprises detecting the detectable moiety and the amount of labeling by a cell-sorting apparatus. In some examples, expressing host cells are determined by measuring relative expression level of the gene product of interest for each genetic variant.

In other embodiments, methods for selecting expressing host cells from a population of host cells having a genetic diversity, the genetic diversity comprising a plurality of genetic variants, wherein at least some of the host cells comprise a polynucleotide sequence encoding a gene product of interest are provided. In some examples, the method includes culturing the population of host cells, whereby the gene product of interest is expressed by a subpopulation of the host cells of the population, the subpopulation thereby comprising expressing host cells, wherein a predetermined property of the expressing host cells varies based on the genetic variant; labeling at least some of the expressing host cells of the subpopulation, wherein the labeling comprises associating the gene product of interest with a detectable moiety, wherein an amount of the labeling proportional to the predetermined property of the gene product of interest in the expressing host cell, thereby producing labeled expressing host cells; and selecting a subset of labeled expressing host cells, wherein the selecting comprises detecting the detectable moiety and the predetermined by a cell-sorting apparatus. In particular examples, the predetermined property of the expressing host cells comprises level of expression of active gene product of interest, level of expression of the gene product of interest, proper protein folding of the gene product of interest, level of expression of properly folded protein of the gene product of interest, cell viability, and/or amount of biomass. In additional examples, expressing host cells are determined by measuring relative expression level of the gene product of interest for each genetic variant.

Also provided are methods for selecting host cells from a population of host cells having genetic diversity of at least 1000, wherein at least some of the host cells comprise a polynucleotide sequence encoding a gene product of interest. In some examples, the methods include culturing the population of host cells, whereby the gene product of interest is expressed by a subpopulation of the host cells of the population, the subpopulation thereby comprising expressing host cells; labeling at least some of the expressing host cells of the subpopulation, wherein the labeling comprises associating the gene product of interest with a detectable moiety, thereby producing labeled expressing host cells; and selecting a subset of labeled expressing host cells, wherein the selecting comprises detecting the detectable moiety by a cell-sorting apparatus. In some examples, expressing host cells are determined by measuring relative expression level of the gene product of interest for each genetic variant.

In embodiments of the disclosed methods, the genetic diversity of the host cell population is host cell genomic variation, polynucleotide sequence variation of one or more expression constructs, or a combination thereof, comprised by at least some of the host cells of the host cell population. In particular examples, the genetic diversity of the population of host cells is 200,000-1,000,000.

In embodiments of the methods, the selecting is fluorescence-activated cell sorting. In some examples, the detectable moiety is a fluorescent moiety and the selecting comprises selecting the 0.01%-5% of cells with highest fluorescence emissions. In a particular non-limiting example, the selecting comprises selecting the 0.5% of cells with highest fluorescence emissions.

In additional embodiments of the methods, the gene product of interest comprises a polypeptide lacking a signal peptide. In other embodiments, the gene product of interest comprises a first polypeptide fused in-frame to a second polypeptide selected from the group consisting of a fluorescent polypeptide and a bioluminescent polypeptide. In some examples, the detectable moiety associated with the gene product of interest comprises the polypeptide selected from the group consisting of a fluorescent polypeptide and a bioluminescent polypeptide. In other embodiments, the gene product of interest comprises a first polypeptide fused in-frame to a second polypeptide having enzymatic activity. In some examples, the detectable moiety associated with the gene product of interest is bound to the active site of the polypeptide having enzymatic activity.

In some embodiments of the methods, the polynucleotide sequence encoding the gene product of interest is an expression vector. In some examples, the expression vector is an extrachromosomal expression vector.

In additional embodiments, labeling at least some of the expressing host cells of the subpopulation comprises fixing the subpopulation of expressing host cells. Fixing the subpopulation of expressing hosts cells may include contacting at least some of the expressing host cells of the subpopulation with an aldehyde, for example paraformaldehyde.

In other embodiments, labeling at least some of the expressing host cells of the subpopulation comprises permeabilizing at least some of the expressing host cells of the subpopulation, for example, contacting at least some of the expressing host cells of the subpopulation with lysozyme.

In further embodiments, labeling at least some of the expressing host cells of the subpopulation further comprises contacting at least some of the expressing host cells of the subpopulation with a compound that labels DNA, for example propidium iodide.

In some embodiments, the population of host cells are prokaryotic cells. In one example, the host cells are Escherichia coli cells, such as E. coli 521 cells.

In some embodiments, the methods also include the recovery of polynucleotides from the subset of labeled expressing host cells, thereby producing recovered polynucleotides. In some examples, the methods also include obtaining DNA sequence information from the recovered polynucleotides. The methods may also further include modifying the genome of a host cell based upon the DNA sequence information, for example, constructing a library of expression vectors based upon the DNA sequence information. In some examples, a parental host cell strain is further transformed with the library of expression vectors. In other examples, the recovered polynucleotides are expression vectors and the methods may further include transforming a parental host cell strain with one or more of the recovered expression vectors. The methods may further include culturing the transformed host cells, wherein at least some of the transformed host cells express the gene product of interest. In some examples, the level of expression of the gene product of interest is determined, for example by gel electrophoresis, enzyme-linked immunosorbent assay (ELISA), liquid chromatography (LC) including high-performance liquid chromatography (HP-LC), solid-phase extraction mass spectrometry (SPE-MS), or an Amplified Luminescent Proximity Homogeneous Assay.

The foregoing and other features of the disclosure will become more apparent from the following detailed description, which proceeds with reference to the accompanying figures.

### BRIEF DESCRIPTION OF THE DRAWINGS

FIGS. 1A-1F is a schematic illustration of an embodiment of the activity-specific cell-enrichment process. The downward-pointing arrow represents the selection of high-performing host cells, starting from a large genetically diverse population of host cells (FIG. 1A), through the application of selective processes represented by the horizontal dashed lines. FIG. 1B indicates selection of high-performing host cells through the use of a cell sorting apparatus, for example by activity-specific cell sorting. FIG. 1C shows the selected population of host cells, which in some embodiments can be the result of transforming the parental host cell strain with extrachromosomal expression vectors recovered from selected high-performing host cells, or with a “high-performance” expression vector library created using sequence information from the selected high-performing host cells. FIGS. 1D and 1E show further selection of high-performing host cells utilizing high-throughput assays such as SPE-MS (FIG. 1D) and/or an activity-based assay (FIG. 1E) such as an antigen-binding assay. As shown in FIG. 1F, the highest-performing host cells can be optimized for both titer and product quality in fermentation processes to ensure scalability. Each of the selection processes shown in FIGS. 1B, 1D, 1E, and 1F can be repeated as needed to further select high-performing host cells.

FIGS. 2A-2C shows three FACS plots indicating the detection events that fall within the ‘low DNA’ gating parameters. In each panel, the DNA fluorescence (675 nm/20 filter FSC-A) of labeled host cells is plotted against the fluorescence (530 nm/30 filter FSC-A) of host cells labeled for TRAST-Fab expression using fluorescently labeled HER2 protein. FIG. 2A is negative control sample A1, a host cell population comprising an empty-vector. FIG. 2B is positive control sample A3, a host cell population expressing TRAST-Fab heavy chain and light chain in a bicistronic arrangement, with cDsbC coexpression (see Table 1). FIG. 2C is experimental sample B1, a host cell population expressing DnaB-TRAST-Fab heavy chain and DnaB-TRAST-Fab light chain in a bicistronic arrangement, with 1.7 million different forms of the expression vector for DnaB-TRAST-Fab present in the host cell population.

FIG. 3 is a histogram showing the results of NGS (next-generation sequencing) analysis of expression vectors recovered from host cells selected by FACS for high levels of DnaB-TRAST-Fab expression. Results are shown for the B1 population of host cells (See Table 1), which comprised expression vectors encoding 137 different gene products that were coexpressed with DnaB-TRAST-Fab from a propionate-inducible (prp) promoter. A sample of the B1 host cells prior to FACS sorting was reserved for NGS analysis, and plasmid DNA from these pre-sort cells and from the FACS-sorted (‘post-sort’) cells was recovered and sequenced by NGS. The identities of the coding sequences coexpressed from the prp promoter were determined from the sequence data, and the frequency at which each of the 137 different gene products was present in the pre-sort and post-sort B1 host cell populations is shown in the histogram.

FIGS. 4A-4B shows two FACS plots indicating the detection events that fall within the ‘low DNA’ gating parameters. In each panel, the DNA fluorescence (675 nm/20 filter FSC-A) of labeled host cells is plotted against the fluorescence (530 nm/30 filter FSC-A) of host cells labeled for TRAST-Fab expression using fluorescently labeled HER2 protein. FIG. 4A is host cell population B1 before sorting by FACS, a host cell population expressing DnaB-TRAST-Fab heavy chain and DnaB-TRAST-Fab light chain in a bicistronic arrangement, with 1.7 million different forms of the expression vector for DnaB-TRAST-Fab present in the host cell population (see Table 1). FIG. 4B is host cell population B1* reconstructed using expression vectors recovered from the B1 host cell population, which was sorted by FACS to select host cells expressing high levels of DnaB-TRAST-Fab.

FIG. 5 is a graph plotting the production of DnaB-TRAST-Fab heavy chain (‘HC’) per host cell culture optical density at 600 nm (‘OD’) against the production of DnaB-TRAST-Fab light chain (‘LC’) per OD, as measured by solid-phase extraction mass spectrometry (SPE-MS). Diverse host cell population B1 was sorted by FACS to identify host cells that expressed high levels of DnaB-TRAST-Fab, and the expression vectors from those high-performing host cells were used to reconstruct a selected host cell population, B1*. Individual B1* host cells were then tested for DnaB-TRAST-Fab expression and the production of DnaB-TRAST-Fab HC and LC peptides were measured by SPE-MS.

FIG. 6 shows FACS plots demonstrating enrichment of Trastuzumab Fab′ high-expressing vectors in three naïve libraries pre-sort (before ACE) and after sorting, isolating the plasmid vector, and retransformation (after ACE). The same sort gate (<0.5%) was applied to both before and after ACE.

FIGS. 7A-7B show FACS plots where gating was established by negative and positive controls (FIG. 7A) and there was an increase in expression of Trastuzumab Fab′ after sorting (FIG. 7B).

### SEQUENCE LISTING

Any nucleic acid and amino acid sequences listed herein or in the accompanying Sequence Listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is the nucleic acid sequence of an exemplary dual-promoter expression vector.

SEQ ID NO: 2 is the amino acid sequence of Trastuzumab-Fab heavy chain A2.

SEQ ID NO: 3 is the amino acid sequence of Trastuzumab-Fab light chain A2.

SEQ ID NO: 4 is the amino acid sequence of a disulfide bond isomerase protein DsbC that is localized to the cell cytoplasm (cDsbC).

SEQ ID NO: 5 is the amino acid sequence of bicistronic Trastuzumab-Fab heavy chain A3.

SEQ ID NO: 6 is the amino acid sequence of bicistronic Trastuzumab-Fab light chain A3.

SEQ ID NO: 7 is the amino acid sequence of Trastuzumab-Fab heavy chain with an N-terminal amino acid sequence derived from Synechocystis sp. DnaB.

SEQ ID NO: 8 is the amino acid sequence of Trastuzumab-Fab light chain with an N-terminal amino acid sequence derived from Synechocystis sp. DnaB.

SEQ ID NO: 9 is the amino acid sequence of an N-terminal amino acid sequence derived from Synechocystis sp. DnaB that includes a 6×His sequence.

### DETAILED DESCRIPTION

The problem of selecting high-performing host cells that can comprise expression constructs from a genetically diverse population of such cells is addressed by the cell-enrichment methods provided herein. These methods provide for the rapid identification and isolation of high-performing host cells, for example, those that express more of the gene product of interest than other host cells present in the genetically diverse host cell population. ‘High-performing’ can also mean expressing less of a gene product of interest, as in cases where it is desirable to identify host cells expressing less of a protease, toxin, or allergenic gene product, for example.

The activity-specific cell-enrichment methods provided identify host cells that express active gene product of interest rather than inactive material. Active gene product can be distinguished from inactive material by the ability of active gene product to specifically bind a binding partner molecule, or by the ability of gene product to participate in a chemical or enzymatic reaction, as examples. The presence of properly formed disulfide bonds in a polypeptide gene product is an indication that it is correctly folded and presumptively active; see Example 1 for methods of determining the locations of disulfide bonds in a polypeptide gene product. In the cell-enrichment methods, active gene product of interest is detected by utilizing an appropriate labeling complex that specifically binds to active gene product of interest, such as a labeled antigen if the gene product of interest is an antibody or Fab; or a labeled ligand if the gene product of interest is a receptor or a receptor fragment, where the ligand specifically binds to an active conformation of the receptor; or a labeled substrate or a labeled substrate analog if the gene product of interest is an enzyme, as examples. For any gene product of interest, if there is an available antibody or antibody fragment that specifically binds to the active gene product and not to inactive gene product, that antibody or antibody fragment can be used to label the active gene product of interest when attached to a detectable moiety, as described below.

Genetic diversity in a population of host cells can result, for example, from genomic variation among the host cells and/or from differences in the polynucleotide sequences of expression constructs comprised by the host cells. If there is genomic diversity among the host cells, selecting high-performing host cells and sequencing genomic DNA recovered from them can be used to identify genomic differences, such as mutations, associated with the superior performance of the selected host cells. If there is diversity between expression constructs in the host cell population, recovering the expression constructs, such as expression vectors, from the selected host cells and sequencing the expression constructs can permit creation of a library of expression constructs (a ‘high-performance library’) that comprises those expression construct elements associated with high-performing host cells. A population of live high-performing host cells can be reconstructed by transforming a parental host cell strain with the high-performance library, or with the recovered high-performing expression constructs themselves. A parental host cell strain can be the strain used to create the host cell population that was screened for high-performing host cells, or another strain that can be genetically modified or transformed with expression constructs to create a host cell strain capable of expressing the gene product of interest.

The activity-specific cell-enrichment methods provided take full advantage of the flow cytometer's speed of sample analysis to isolate high-performing host cells, such as those that express more gene product of interest. In some embodiments, populations of host cells over one million in diversity can be analyzed within minutes to determine whether a higher-performing subset population exists. If so, and if the flow cytometer is a FACS instrument, several hundred higher-performing host cells from a rare (one in one million) subpopulation can be isolated within an hour to enable subsequent analysis. The criteria that define subpopulations of host cells can include none, some, or all the host cells of the population within the defined subpopulation; in some instances, the subpopulation may be coextensive with the population. For example, a subpopulation of host cells, defined by expression of a labeled gene product of interest at levels detectable by a flow cytometer, can include all—or a substantial majority of—the host cells of the population.

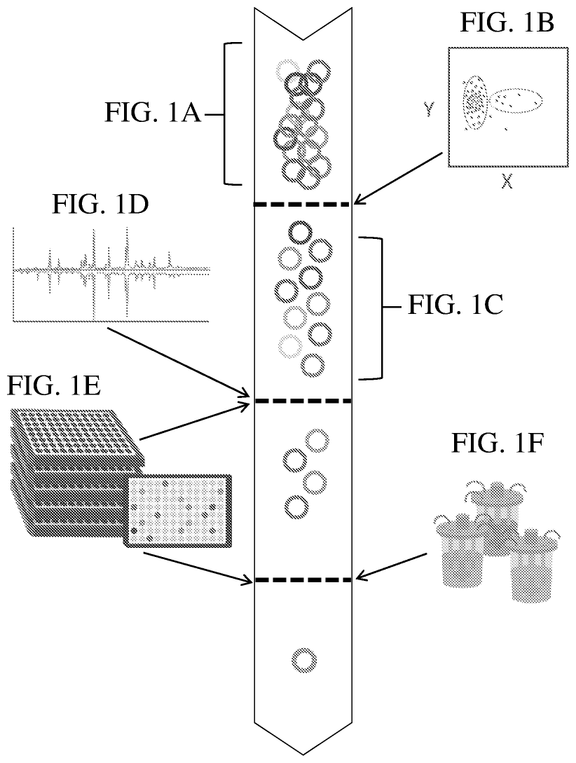

The activity-specific cell-enrichment methods in some embodiments involve the following aspects: (1) providing a genetically diverse population of host cells that can comprise expression constructs; (2) labeling the gene product of interest within the host cells by expressing the gene product of interest as a detectable fusion protein, or by contacting the gene product of interest with a labeling complex that specifically binds the active gene product of interest; (3) selecting high-performing host cells using a sorting apparatus that employs flow cytometry or a comparable method; (4) analyzing the selected host cells and/or expression vectors; (5) reconstructing host cell strains; (6) optionally further analyzing reconstructed host cell strains, particularly with respect to the activity of the gene product of interest; and (7) optionally repeating any or all of (1)-(6) above. These aspects of the methods are shown schematically in FIGS. 1A-1F and described in additional detail below.

I. Genetically Diverse Populations of Host Cells and/or Expression Constructs

A. Host Cells

The cell-enrichment methods disclosed herein are designed to select host cells expressing desired levels of active gene product. For use in the cell-enrichment methods described herein, host cells can be any cell capable of expressing gene product and being sorted by flow cytometry or a comparable method, such as single-celled organisms, isolated cells grown in culture, or isolated cells derived from a multicellular organism. Examples of host cells are provided that allow for efficient inducible expression of gene products, such as polypeptide gene products that comprise disulfide bonds.

Particularly suitable host cells are capable of growth at high cell density in fermentation culture, and can produce gene products in oxidizing host cell cytoplasm through highly controlled inducible gene expression. Host cells with these qualities are produced by combining some or all of the following characteristics. (1) The host cells are genetically modified to have an oxidizing cytoplasm, through increasing the expression or function of oxidizing polypeptides in the cytoplasm, and/or by decreasing the expression or function of reducing polypeptides in the cytoplasm. Increased expression of the cysteine oxidase DsbA, the disulfide isomerase DsbC, or combinations of the Dsb proteins, which are all normally transported into the periplasm, has been utilized in the expression of heterologous proteins that require disulfide bonds (Makino et al., “Strain engineering for improved expression of recombinant proteins in bacteria”, Microb Cell Fact 2011 May 14; 10: 32). It is also possible to express cytoplasmic forms of these Dsb proteins, such as a cytoplasmic version of DsbC (‘cDsbC’), for example having an N-terminal truncation of twenty amino acids, which lacks a signal peptide and therefore is not transported into the periplasm. Cytoplasmic Dsb proteins such as cDsbC are useful for making the cytoplasm of the host cell more oxidizing and thus more conducive to the formation of disulfide bonds in heterologous proteins produced in the cytoplasm. The host cell cytoplasm can also be made more oxidizing by altering the thioredoxin and the glutaredoxin/glutathione enzyme systems directly: mutant strains defective in glutathione reductase (gor) or glutathione synthetase (gshB), together with thioredoxin reductase (trxB), render the cytoplasm oxidizing. These strains are unable to reduce ribonucleotides and therefore cannot grow in the absence of exogenous reductant, such as dithiothreitol (DTT). Suppressor mutations (ahpC* or ahpC^{Δ}) in the gene ahpC, which encodes the peroxiredoxin AhpC, convert it to a disulfide reductase that generates reduced glutathione, allowing the channeling of electrons onto the enzyme ribonucleotide reductase and enabling the cells defective in gor and trxB, or defective in gshB and trxB, to grow in the absence of DTT. A different class of mutated forms of AhpC can allow strains, defective in the activity of gamma-glutamylcysteine synthetase (gshA) and defective in trxB, to grow in the absence of DTT; these include AhpC V164G, AhpC S71F, AhpC E173/S71F, AhpC E171Ter, and AhpC dup162-169 (Faulkner et al., “Functional plasticity of a peroxidase allows evolution of diverse disulfide-reducing pathways”, Proc Natl Acad Sci USA 2008 May 6; 105(18): 6735-6740, Epub 2008 May 2). (2) Optionally, host cells can also be genetically modified to express chaperones and/or cofactors that assist in the production of the desired gene product(s), and/or to glycosylate polypeptide gene products. (3) The host cells contain additional genetic modifications designed to improve certain aspects of gene product expression from the expression construct(s). In particular embodiments, the host cells (A) have an alteration of gene function of at least one gene encoding a transporter protein for an inducer of at least one inducible promoter, and as another example, wherein the gene encoding the transporter protein is selected from the group consisting of araE, araF, araG, araH, rhaT, xylF, xylG, and xylH, or particularly is araE, or wherein the alteration of gene function more particularly is expression of araE from a constitutive promoter; and/or (B) have a reduced level of gene function of at least one gene encoding a protein that metabolizes an inducer of at least one inducible promoter, and as further examples, wherein the gene encoding a protein that metabolizes an inducer of at least one inducible promoter is selected from the group consisting of araA, araB, araD, prpB, prpD, rhaA, rhaB, rhaD, xylA, and xylB; and/or (C) have a reduced level of gene function of at least one gene encoding a protein involved in biosynthesis of an inducer of at least one inducible promoter, which gene in further embodiments is selected from the group consisting of scpA/sbm, argK/ygfD, scpB/ygfG, scpC/ygfH, rmlA, rmlB, rmlC, and rmlD.

In certain embodiments, the host cells are microbial cells such as yeasts (Saccharomyces, Schizosaccharomyces, etc.) or bacterial cells, or are gram-positive bacteria or gram-negative bacteria, or are E. coli, or are an E. coli B strain, or are E. coli B strain 521 cells, or are E. coli B strain 522 cells. E. coli 521 and 522 cells have the following genotypes:
E. coli 521: ΔaraBAD fhuA2 [lon] ompT ahpC^{Δ} gal λatt::pNEB3-r1-cDsbC (Spec, lacI) ΔtrxB sulA11R(mcr-73::miniTn10—Tet^{S})2 [dcm] R(zgb-210::Tn10—Tet^{S}) ΔaraEp::J23104 ΔscpA-argK-scpBC endA1 rpsL-Arg43 Δgor Δ(mcrC-mrr)114::IS10
E. coli 522: ΔaraBAD fhuA2 prpD [lon] ompT ahpC^{Δ} gal λatt::pNEB3-r1-cDsbC (Spec, lacI) ΔtrxB sulA11R(mcr-73::miniTn10—Tet^{S})2 [dcm] R(zgb-210::Tn10—Tet^{S}) ΔaraEp::J23104 ΔscpA-argK-scpBC endA1 rpsL-Arg43 Δgor Δ(mcrC-mrr)114::IS10

In growth experiments with E. coli host cells having oxidizing cytoplasm, we have determined that E. coli B strains with oxidizing cytoplasm are able to grow to much higher cell densities than a corresponding E. coli K strain. Other suitable strains include E. coli B strains SHuffle® Express (NEB Catalog No. C3028H) and SHuffle® T7 Express (NEB Catalog No. C3029H), and the E. coli K strain SHuffle® T7 (NEB Catalog No. C3026H).

In some embodiments, the host cells are prokaryotic host cells. Prokaryotic host cells can include archaea (such as Haloferax volcanii, Sulfolobus solfataricus), Gram-positive bacteria (such as Bacillus subtilis, Bacillus licheniformis, Brevibacillus choshinensis, Lactobacillus brevis, Lactobacillus buchneri, Lactococcus lactis, and Streptomyces lividans), or Gram-negative bacteria, including Alphaproteobacteria (Agrobacterium tumefaciens, Caulobacter crescentus, Rhodobacter sphaeroides, and Sinorhizobium meliloti), Betaproteobacteria (Alcaligenes eutrophus), and Gammaproteobacteria (Acinetobacter calcoaceticus, Azotobacter vinelandii, Escherichia coli, Pseudomonas aeruginosa, and Pseudomonas putida). Preferred host cells include Gammaproteobacteria of the family Enterobacteriaceae, such as Enterobacter, Erwinia, Escherichia (including E. coli), Klebsiella, Proteus, Salmonella (including Salmonella typhimurium), Serratia (including Serratia marcescans), and Shigella.

Many additional types of host cells can be used in the methods provided herein, including eukaryotic cells such as yeast (Candida shehatae, Kluyveromyces lactis, Kluyveromyces fragilis, other Kluyveromyces species, Pichia pastoris, Saccharomyces cerevisiae, Saccharomyces pastorianus also known as Saccharomyces carlsbergensis, Schizosaccharomyces pombe, Dekkera/Brettanomyces species, and Yarrowia lipolytica); other fungi (Aspergillus nidulans, Aspergillus niger, Neurospora crassa, Penicillium, Tolypocladium, Trichoderma reesia); insect cell lines (Drosophila melanogaster Schneider 2 cells and Spodoptera frugiperda Sf9 cells); and mammalian cell lines including immortalized cell lines (Chinese hamster ovary (CHO) cells, HeLa cells, baby hamster kidney (BHK) cells, monkey kidney cells (COS), human embryonic kidney (HEK, 293, or HEK-293) cells, and human hepatocellular carcinoma cells (Hep G2)). The above host cells are available from the American Type Culture Collection.

B. Expression Constructs

Expression constructs are polynucleotides designed for the expression of one or more gene products of interest. Certain gene products of interest are heterologous gene products, in that they are derived from species that are different from that of the host cell in which they are expressed, and/or are heterologous gene products that are not natively expressed from the promoter(s) utilized within the expression construct. Gene products of interest include modified gene products that have been designed to include differences from naturally occurring forms of such gene products. Examples of heterologous and/or modified gene products include polypeptide gene products lacking a signal peptide, that are therefore expressed and retained within the host cell cytoplasm. Expression constructs comprising polynucleotides encoding heterologous and/or modified gene products, or comprising a combination of polynucleotides that were derived from organisms of different species, or comprising polynucleotides that have been modified to differ from naturally occurring polynucleotides, are not naturally occurring molecules. Expression constructs can be integrated into a host cell chromosome, or maintained within the host cell as polynucleotide molecules replicating independently of the host cell chromosome, such as plasmids or artificial chromosomes. An example of an expression construct is a polynucleotide resulting from the insertion of one or more polynucleotide sequences into a host cell chromosome, where the inserted polynucleotide sequences alter the expression of chromosomal coding sequences. An expression vector is a plasmid expression construct specifically used for the expression of one or more gene products. One or more expression constructs can be integrated into a host cell chromosome or be maintained on an extrachromosomal polynucleotide such as a plasmid or artificial chromosome. Suitable expression constructs include the dual-promoter expression vectors described in United States patent application publication US20160376602A1, which is incorporated by reference herein.

Expression constructs such as extrachromosomal expression vectors can comprise an origin of replication, such as colE1, pMB1 (pBR3220), modified pMB1 (pUC9), R1(ts) (pMOB45), p15A (pPRO33), pSC101, RK2, CloDF13 (pCDFDuet™-1), ColA (pCOLADuet™-1), and RSF1030/NTP1 (pRSFDuet™-1). Expression constructs can also comprise at least one selectable marker that confers antibiotic resistance, such as ampicillin (Amp®), chloramphenicol (Cml® or Cm®), kanamycin (Kan®), spectinomycin (Spc®), streptomycin (Str®), and tetracycline (Tet®). Further, expression constructs can comprise a multiple cloning site (MCS), also called a polylinker, which is a polynucleotide that contains multiple restriction sites in close proximity to or overlapping each other. The restriction sites in the MCS typically occur once within the MCS sequence, and preferably do not occur within the rest of the plasmid or other polynucleotide construct, allowing restriction enzymes to cut the plasmid or other polynucleotide construct only within the MCS.

Examples of MCS sequences include those in the pBAD series of expression vectors, such as pBAD24 and pBAD33 (Guzman et al., “Tight regulation, modulation, and high-level expression by vectors containing the arabinose PBAD promoter”, J Bacteriol 1995 July; 177(14): 4121-4130), and those in the pPRO series of expression vectors derived from the pBAD vectors, such as pPRO33 (U.S. Pat. No. 8,178,338).

For expression constructs encoding at least one polypeptide gene product, the polynucleotide region between the transcription initiation site and the initiation codon of the coding sequence of the polypeptide gene product that is to be expressed corresponds to the 5′ untranslated region (‘UTR’) of the mRNA for that polypeptide gene product. Preferably, the region of the expression construct that corresponds to the 5′ UTR comprises a nucleotide sequence similar to the consensus ribosome binding site (RBS, also called the Shine-Dalgarno sequence) that is found in the species of the host cell. In prokaryotes (archaea and bacteria), the RBS consensus sequence comprises the nucleotide sequence GGAGG or GGAGGU, and in bacteria such as E. coli, the RBS consensus sequence is AGGAGG or AGGAGGU. The RBS is typically separated from the initiation codon by 5 to 10 intervening nucleotides, and is often located in very close proximity 5′ to (or ‘upstream of’) of the MCS within expression constructs.

For the efficient expression of one or more gene products, expression constructs preferably comprise at least one promoter, such as a constitutive or an inducible promoter, and preferably an inducible promoter. Within an expression construct, a promoter is placed upstream of any RBS sequence and of the coding sequence for the gene product that is to be expressed, so that the presence of the promoter will direct transcription of the gene product coding sequence in a 5′ to 3′ direction relative to the coding strand of the polynucleotide encoding the gene product. Examples of inducible promoters that can be used in expression constructs are the well-known E. coli sugar-inducible promoters, such as the L-arabinose-inducible promoter ParaBAD, the lactose-inducible promoter PlacZYA, the rhamnose-inducible promoter PrhaBAD, and the xylose-inducible promoters PxylAB and PxylFGHR; the E. coli propionate-inducible promoter PprpBCDE; and the promoter inducible by phosphate depletion PphoA, all of which are described in detail in PCT application publication WO2016205570A1, which is incorporated by reference herein. Constitutive promoters such as the J23104 promoter can be obtained from the Registry of Standard Biological Parts maintained by iGEM (Boston, Mass.); see parts.igem.org/Promoters/Catalog.

C. Host Cell Population Genetic Diversity

The provided methods are advantageously used to select high-performing host cells from a genetically diverse population of host cells, in which the diversity or variation within the host cell population can arise for example from differences between host cell genomes, or between expression constructs comprised by the host cells. The host cell population genetic diversity can be randomly generated by processes such as mutation, or specifically introduced by targeted methods of making changes in the host cell genome or in expression constructs, which are then introduced into the host cell strain.

The host cell population comprises a plurality of genetic variants. In many embodiments, one aspect of the present invention comprises sorting a host cell population based on a predetermined property of the host cells, which predetermined property varies based on the genetic variants within the host cell population. In many embodiments, the predetermined property is the expression level of a gene product of interest, and the methods include detecting the expression levels of an active gene product of interest within each of the plurality of genetic variants. Additional predetermined properties of the host cells include expression level of active gene product of interest, proper folding of the gene product of interest, expression level of properly folded protein, cell viability, and/or biomass. The genetic diversity of the host cell population should therefore comprise a plurality of genetic variants, which genetic variants are sufficiently numerous to provide for variations in expression levels or other predetermined properties within the genetically diverse population. In some embodiments, the number of genetic variants capable of substantially expressing a gene product of interest may be very small, which may require increasing the genetic diversity. In many embodiments, the genetic diversity of the host cell population may be increased as described herein until a suitable genetic diversity is achieved.

In embodiments of the disclosed methods, the genetic diversity of the host cell population is defined as the number of different genetic variants present in the host cell population, the number of different genetic variants relative to a negative control, and/or the number of different genetic variants relative to a reference cell strain. The number of genetic variants may be the actual number of variants or a calculated (“target”) number of genetic variants in the host cell population. These variants may be the result of one or more genetic (e.g., nucleic acid sequence) differences in the host cell genome between cells, one or more genetic (e.g., nucleic acid sequence) differences in expression construct(s) between host cells, or a combination thereof. In some examples, the genetic differences include alteration, deletion, or insertion of one or more nucleotides of a sequence or insertion or deletion of one or more elements (such as one or more tags, domains, expression control sequences, and/or associated proteins).

In some embodiments, the genetic diversity of the host cell population is at least 500, at least 1000, at least 2000, at least 5000, at least 10,000, and least 50,000, at least 100,000, at least 200,000, at least 500,000, at least 1,000,000, at least 2,000,000, at least 5,000,000, at least 10,000,000, at least 100,000,000, at least 500,000,000, or at least 1,000,000,000. In other examples, the genetic diversity is about 1000-1,000,000,000, such as about 1000-10,000, about 5000-50,000, about 50,000-200,000, about 100,000-500,000, about 200,000-1,000,000, about 500,000-2,000,000, about 1,000,000-5,000,000, about 5,000,000-50,000,000, about 20,000,000-100,000,000, about 50,000,000-500,000,000, or about 500,000,000-1,000,000,000.

Any type of genetic diversity can be probed using the methods provided herein. In some embodiments, the genetic diversity includes one or more of differences (including alteration or presence or absence) between a gene product of interest (including but not limited to coding sequence variants and codon-optimization), promoters (including constitutive and/or inducible promoters), chaperones, ribosome binding sequences, tags, nuclear localization signals, signal peptides, knockout or knockin of one or more genes, presence of one or more (such as 1, 2, 3, or more) plasmids, or any combination thereof. In some examples, the genetic diversity is generated by standard directed genetic modification techniques. In other examples, the genetic diversity is generated by random mutagenesis, error-prone PCR mutagenesis, or transposon mutagenesis (e.g., Tn5). A combination of techniques can also be used to generate additional levels of genetic diversity.

There are many methods known in the art for making alterations to host cell genomes or expression constructs in order to change nucleotide sequences and/or to eliminate, reduce, or change gene function. Methods of making targeted disruptions of genes in host cells such as E. coli and other prokaryotes have been described (Muyrers et al., “Rapid modification of bacterial artificial chromosomes by ET-recombination”, Nucleic Acids Res 1999 Mar. 15; 27(6): 1555-1557; Datsenko and Wanner, “One-step inactivation of chromosomal genes in Escherichia coli K-12 using PCR products”, Proc Natl Acad Sci USA 2000 Jun. 6; 97(12): 6640-6645), and kits for using similar Red/ET recombination methods are commercially available (for example, the Quick & Easy E. coli Gene Deletion Kit from Gene Bridges GmbH, Heidelberg, Germany). Red/ET recombination methods can also be used to replace a promoter sequence with that of a different promoter, such as a constitutive promoter, or an artificial promoter that is predicted to promote a certain level of transcription (De Mey et al., “Promoter knock-in: a novel rational method for the fine tuning of genes”, BMC Biotechnol 2010 Mar. 24; 10: 26). The function of host cell genomes or expression constructs can also be eliminated or reduced by RNA silencing methods (Man et al., “Artificial trans-encoded small non-coding RNAs specifically silence the selected gene expression in bacteria”, Nucleic Acids Res 2011 April; 39(8): e50, Epub 2011 Feb. 3). The Gibson assembly method (Gibson, “Enzymatic assembly of overlapping DNA fragments”, Methods Enzymol 2011; 498: 349-361; doi: 10.1016/B978-0-12-385120-8.00015-2) can also be used to make targeted changes in host cell genomes or expression constructs, such as insertions, deletions, and point mutations. Another method for making directed alterations in host cell genomes or expression constructs utilizes CRISPR (clustered regularly interspaced short palindromic repeats) nucleotide sequences and Cas9 (CRISPR-associated protein 9), which recognizes and cleaves nucleotide sequences that are complementary to CRISPR sequences. Further, changes to host cell genomes can be introduced through traditional genetic methods.

II. Labeling Gene Product within Host Cells

Labeling the gene product of interest involves the association of the gene product of interest with a detectable moiety. The association of the gene product of interest with a detectable moiety can occur in different ways, including but not limited to: a covalent bond between the gene product of interest and the detectable moiety, as when the gene product of interest is a polypeptide expressed as a fusion polypeptide with a detectable fluorescent or luminescent polypeptide; a non-covalent binding interaction, as between an antibody gene product of interest and an antigen; or an association between expression of the gene product of interest and a detectable change in the host cell, such as a change in intracellular calcium concentration caused by expression of the gene product of interest.

For selecting live host cells by cell sorting, where the host cells are expressing a gene product of interest in the cytoplasm, it is necessary to label the gene product within the cytoplasm so that a detectable signal is associated with that particular host cell. In some examples, where the gene product of interest has enzymatic activity, it is possible to introduce a cell-permeable chromogenic substrate for that enzyme into the cell. In other examples, if the presence of active gene product of interest is correlated with another attribute of the host cell that can be detected without killing the host cell, such as measuring the intracellular calcium concentration using a fluorescent reporter protein like aequorin, the host cell can be genetically modified to include a reporter protein or other molecule.

As another example, the host cells comprise expression constructs encoding the polypeptide(s) of interest as fusion proteins, at least one of which has a fluorescent protein such as green fluorescent protein (GFP) expressed in frame at its N- or C-terminus, and preferably at its C-terminus. Such fusion proteins can also comprise a linker polypeptide between the amino acid sequence of each polypeptide of interest and the fluorescent protein. Preferably, the polynucleotide sequence encoding the fluorescent portion of the polypeptide(s) of interest can be easily removed from the expression vectors by digestion with one or more restriction enzymes. If the gene product of interest comprises more than one polypeptide chain, such as an antibody comprising a heavy chain and a light chain, two or more of the constituent polypeptides can each be fused to one component of BRET (bioluminescence resonance energy transfer) or FRET (fluorescence resonance energy transfer) donor/acceptor pair, so that a fluorescent signal is generated by expression and assembly of the constituent polypeptides and association of the BRET or FRET donor and acceptor, providing a measure of both expression quantity and the ability of the constituent polypeptides to form the gene product of interest.

In some instances, the expression of one or more polypeptides of interest as a fusion with a fluorescent or luminescent protein might affect the folding, conformation, and/or activity of the polypeptide(s) of interest, but even in this case a FACS selection based on the amount of fluorescence or luminescence can identify live host cells that express the desired amount of the polypeptide(s) of interest. For example, if a BRET donor and acceptor are expressed as fusion polypeptides with polypeptide components of the gene product of interest, but the BRET donor and acceptor cannot achieve the requisite proximity for the BRET acceptor to produce a signal, a FACS selection can be performed by detecting the BRET donor bioluminescence.

In some embodiments, the activity-specific cell-enrichment methods can also involve the labeling of host cells by labeling complexes that specifically interact with active gene product of interest. Labeling complexes can also include polypeptide or other chemical linkers to connect components of the labeling complex to each other, or to connect the labeling complex to cellular structures, or to extend to or beyond the cell surface for attachment to beads or other media that are helpful for detection or purification. For gene products of interest that are expressed and retained in the host cell cytoplasm, the labeling procedure can include fixation, so that the gene product of interest produced by the host cell will remain in association with the particular host cell that produced it, and permeabilization of the host cells, so that the labeling complexes will be able to access the gene product of interest.

Labeling Complexes. For use in the activity-specific cell-enrichment methods, the labeling complexes can include a component that provides specificity for the active gene product of interest, and the presence of a detectable moiety. A detectable moiety produces an emission of light, electromagnetic radiation, and/or particles that is detectable by the sorting apparatus, allowing for the selection of high-performing host cells.

The specificity of the labeling complex for active gene product can be established by using a binding partner (or “specificity component”) that only binds to active gene product, such as an antigen to label an antibody or antibody fragment, a ligand (specific for active receptor) to label a receptor or receptor fragment, a substrate or substrate analog molecule to label an enzyme, or an antibody or antibody fragment specific for active gene product to label that gene product. As an example, if the gene product of interest is an antibody, three separate labeling complexes could be used, individually or in any combination, to detect active antibody gene product: labeled antigen to specifically bind the antigen-binding domain, labeled anti-Fc antibody to specifically bind properly folded and/or assembled Fc region, and labeled anti-light-chain antibody to specifically bind properly folded and/or assembled light chain. As an additional example, if the gene product comprises a polyribonucleotide, the specificity of the labeling complex can be provided by a polynucleotide that specifically binds to the polyribonucleotide under the conditions of the labeling reaction.

The detectable moiety of the labeling complex can comprise a chromophore, a fluorophore, and/or a luminophore, in each case producing a detectable change in absorbance of light, or a light emission, under certain conditions. An example of a suitable fluorescent detectable moiety is streptavidin-Alexa Fluor® 488 (ThermoFisher Scientific Inc., Waltham, Mass.). The detectable moiety of the labeling complex can also comprise a radioactive isotope that generates emissions detectable by scintillation or by direct beta or gamma ray detection, if the apparatus to be used to sort the labeled host cells is capable of detecting and utilizing the radioactive emissions. A further type of detectable moiety can comprise one or more atoms of a heavy metal (for example, iron, nickel, copper, zinc, gallium, ruthenium, silver, cadmium, indium, tin, hafnium, platinum, gold, mercury, thallium, or lead), so that the presence or absence of the detectable moiety can be detected by a mass spectrometer. Another example of a detectable moiety is one that is associated with a magnetic field that can be detected by a sorting apparatus. In the case where the gene product of interest is an enzyme, the detectable moiety can comprise a fluorescent molecule attached to a substrate analog, which will bind specifically to the active site of the enzyme. As another example, if the gene product of interest is an enzyme and the detectable moiety is associated with the substrate of the enzyme, the apparatus to be used to sort the labeled host cells can be set to detect a change in the absorbance, fluorescence, or luminescence produced by the detectable moiety: either a decrease in those cases where the signal from the detectable moiety is reduced when the substrate is converted by the enzyme, or an increase in those cases where the signal from the detectable moiety becomes detectable as a result of enzymatic conversion of the substrate. As a particular example, a chromogenic enzyme substrate can provide specificity as a labeling complex, in that it interacts specifically with the active site of the enzyme, and is also the detectable moiety of a labeling complex, in that it generates a detectable change in absorbance of light as a result of interaction with the enzymatic gene product of interest. One such chromogenic enzyme substrate is Chromogenix S-2222™ (Diapharma, West Chester, Ohio), which binds to and is cleaved by the serine endopeptidase Factor Xa, activating the chromophore para-nitroaniline (pNA).

In some cases the specificity component of the labeling complex—antigen, ligand, substrate, substrate analog, antibody, etc. —is commercially available as a conjugate with a chromophore or other type of detectable moiety. In other cases the specificity component is commercially available as a conjugate with a covalently linked binding moiety, such as biotin, and this conjugate can be bound to a detectable moiety covalently linked to the binding partner of the binding moiety, such as streptavidin. An example of a suitable conjugate comprising a binding moiety and a detectable moiety is streptavidin-Alexa Fluor® 488 (ThermoFisher Scientific Inc., Waltham, Mass.). In situations where no such conjugates are commercially available, a binding moiety such as biotin can be conjugated to the specificity component of the labeling complex. Other binding moiety-binding partner pairs that can be used include the inclusion of a poly-histidine amino acid sequence, a run of six or more histidines, preferably six to ten histidine residues, in a polypeptide specificity component of the labeling complex, and binding that to a nickel- or cobalt-conjugated detectable moiety. Another example of a binding-moiety-binding-partner pair is the SpyTag-SpyCatcher pair: SpyTag is a peptide of 13 amino acids that is bound by the 12.3-kDa SpyCatcher protein, forming a covalent intermolecular isopeptide bond.

As an additional example, the specificity component (for example, the HER2 antigen) could be bound by an antibody (for example, anti-HER2 secondary antibody) as a binding moiety, conjugated to a detection moiety, where the antibody specifically recognizes the specificity component in a manner that does not interfere with the binding between the specificity component and the gene product of interest. In further variations of this arrangement, the detection moiety can be conjugated to an antibody that specifically recognizes the antibody that specifically recognizes the specificity component, and so on, as long as each antibody in the chain is specific for its binding target.

There are also several ‘split protein’ or ‘protein fragment complementation’ binding pairs, where the separately expressed domains of a protein have affinity for each other, and when the domains bind, an activity of the split protein is restored. For example, using one domain of beta lactamase, beta galactosidase, horseradish peroxidase, or luciferase as the binding moiety and a complementary domain of the same protein as the binding partner will reconstitute an enzyme that can generate a detectable signal when its substrate is present, and in some particular examples, the substrate can be provided as part of a fusion protein with one or more of the binding domains. In another example, termed bimolecular fluorescence complementation (BiFC), the ‘split’ protein is a fluorescent protein, such as green fluorescent protein or yellow fluorescent protein, that can be separated into protein fragments each attached by a linker to a member of a complementary binding pair, such as an anti-parallel leucine zipper motif. When reassociated through interaction of the leucine zipper motif, the fluorescent protein activity is restored, creating a detectable moiety.

One method that can be used for specific labeling and also for detection is the Alpha (Amplified Luminescent Proximity Homogeneous Assay) technology (PerkinElmer, Waltham Mass.), in which the binding of two binding partners—for example, a gene product of interest and a specificity component—brings a donor bead (attached to one binding partner) and an acceptor bead (attached to the other binding partner) into proximity, so that excitation of the donor bead at one wavelength (680 nm) will result in a chemical energy transfer to the acceptor bead and emission at a different wavelength (520-620 nm). In this technology, the donor bead and the acceptor bead create a detectable moiety when brought into proximity.

Fixation. The gene product of interest can be retained within the host cells by fixing the host cells with a crosslinking reagent, such as one or more aldehydes (paraformaldehyde, glutaraldehyde, formaldehyde), applied in solution. Fixation of the gene product of interest within the host cells using one or more aldehydes is an example of electrophile/nucleophile chemistry, where the aldehydes are the electrophiles and the gene product of interest supplies the nucleophilic centers, such as the amine groups in polypeptides and the N7-position of guanine residues of polynucleotides. Crosslinking reagents are typically bifunctional and can react with the gene product of interest at one end, and with a component of the host cell (DNA, RNA, cytoskeleton, membrane, cell wall, or protein complexed to one of these components) at the other end. Many different types of crosslinking reagents are commercially available (ThermoFisher Scientific Inc., Waltham, Mass.). Another method of retaining the gene product of interest within the host cell involves including a polynucleotide sequence encoding a polypeptide or polynucleotide that associates with a structure of the host cell, such as a cytoskeletal component or other cytoplasmic structure, within the coding sequence for the gene product of interest. For example, particularly in prokaryotic host cells, attaching all or part of the cytoskeletal MreB protein or its analog to a gene product of interest can cause the gene product of interest to become associated with the inner cell membrane through the interaction of MreB with MreC or an analogous protein.

Permeabilization. The host cells are permeabilized by treatment with lysozyme and EDTA, or with lysozyme and a detergent such as octylglucoside to facilitate lysozyme penetration.

Labeling the Nucleic Acids of Host Cells. The DNA and other nucleic acids of live host cells can be labeled with dyes that are uncharged (such as Hoechst 33342) or that contain conjugated systems to distribute any charge, making them able to permeate cells. However, a live host cell may transport dye back out of the cell. Host cells can be fixed and/or permeabilized to allow DNA-labeling compound(s) to enter and remain in the host cells. Compounds that label DNA in fixed cells include propidium iodide (PI), 7-aminoactinomycin-D (7-AAD), and 4′6′-diamidino-2-phenylindole (DAPI). Thus, in some examples, a DNA stain is utilized to identify live cells in the population.

### III. Selecting High-Performing Host Cells

The labeled host cell population is sorted using an apparatus capable of detecting the emissions (light, electromagnetic radiation, etc.) produced by each labeled host cell, and sorting each host cell on the basis of factors such as the amount of the emissions detected for that cell. A sorting apparatus can utilize any type of cell-sorting technology, such as flow cytometry or microfluidic cell sorting, which can sort cells one at a time by a use of a laser detector. In MACS (magnetically activated cell sorting) the host cells are labeled with a magnetic particle, and in affinity-based cell sorting, the host cells are labeled with a labeling complex that extends to or beyond the cell surface for affinity-based interaction with solid media such as a resin. The MACS and affinity-based cell-sorting technologies do not isolate single cells, but can group host cells based on levels of specific binding of labeling complexes to gene products of interest within host cells.

In some embodiments, the methods include sorting a population of host cells including at least 200 cells. For example, the population of host cells may include at least 200 cells, at least 500 cells, at least 1000 cells, at least 2000 cells, at least 5000 cells, at least 10,000 cells, at least 20,000 cells, at least 40,000 cells, at least 50,000 cells, at least 75,000 cells, at least 100,000 cells, at least 200,000 cells, at least 500,000 cells, or more. In one example, the population of host cells that is sorted includes 200-40,000 cells. However, one of ordinary skill in the art will understand that any number of cells may be sorted, provided sufficient time and equipment capacity, and the number of selected cells provides sufficient DNA for subsequent steps.

In one embodiment, the sorting apparatus utilizes flow cytometry. Flow cytometry is a powerful technology for the analysis of a population of cells, having the ability to simultaneously measure multiple parameters at the single-cell level at high speeds (100,000 or more events (cells) per second). A flow cytometer typically operates by (1) separating each individual cell in the population, (2) sequentially irradiating (or interrogating”) each cell with one more laser(s), and (3) recording the emitted light associated with that irradiated cell. A flow cytometer equipped with the ability to sort cells into two or more containers, one cell at a time, based on the emitted light associated with a given cell, is called a Fluorescence-Activated Cell Sorter (FACS). FACS instruments allow isolation of one or more specific cell type(s) from a complex population for subsequent analysis. An example of a suitable FACS instrument is the BD FACSAria™-IIu (Becton, Dickinson and Co., Franklin Lakes, N.J.).

In a FACS instrument, a population of cells, such as labeled host cells, is funneled through a nozzle that creates a single-cell stream that then flows past a set of laser light sources, one cell at a time. Host cells labeled with an appropriate detectable moiety such as a fluorophore are detected by a distinct fluorescent signal generated by excitation or emission or both. When interrogated by the lasers, the cell scatters light that is measured by two optical detectors. One detector measures scatter along the path of the laser; this parameter is referred to as forward scatter (FSC). The measurement of forward scatter allows for the discrimination of cells by size, because FSC intensity is proportional to the diameter of the cell, and is primarily due to light diffraction around the cell. The other detector measures scatter at a ninety-degree angle relative to the laser; this parameter is called side scatter (SSC). Side scatter measurement provides information about the internal complexity (“granularity”) of a cell. The interaction between the laser and intracellular structures causes the light to refract or reflect. For each cell, the FACS instrument measures each of FSC and SSC as a ‘pulse’ that can be visualized as a curve having a width (W), a height (H), and an area (A) under the curve. When measured in conjunction, the FSC and SSC measurements for each cell allow for some degree of differentiation between cells within a heterogeneous population. Some commonly measured parameters of cells include cell size and granularity as described above, and target protein abundance and/or DNA content when the target protein(s) and/or DNA are detectably labeled.

To provide a benchmark for comparison of fluorescence measurements, labeled host cells from a control host cell strain that has been characterized for levels of expression of the gene product of interest, preferably levels of active gene product of interest, can be scanned by FACS. The FSC and/or SSC of the control host cell strain can be measured at certain settings of the FACS apparatus, for example at particular voltages for the photomultiplier tubes (PMTs). When an experimental sample, such as a highly genetically diverse host cell population expressing the gene product of interest, is scanned by FACS using the same settings of the FACS apparatus as were used for the control host cell strain, the resulting FSC and/or SSC reading can be compared to that reading obtained for the control host cell strain, to see if the experimental sample is likely to yield higher-performing host cells than the ‘benchmark’ control host cell strain. In some embodiments, a control host cell strain is a negative control, such as a host cell strain that does not express the gene product of interest in the experimental sample.

Gating. Gating is the process of setting selection ranges within the parameter(s) that have been selected for measurement, where cells that exhibit characteristics within the selection ranges will be selected and sorted away from non-selected cells. The gating parameters can often be visualized as a defined region on a FACS plot having one, two, three, or more dimensions. For example, gating parameters can be visualized as a defined area on a two-dimensional plot of fluorescence measured as SSC-W against fluorescence measured as FSC-H, to select detection events falling within that defined area, at the range of SSC-W values consistent with the fluorescence from a single cell. In particular examples, the gating parameters also identify and eliminate aggregated cells or non-cellular debris, in order to measure signal substantially only from single cells. This reduces artifacts of increased expression of the product of interest due to cell “clumping” rather than actual increase due to the particular genetic diversity of a cell.

IV. Analyzing the Selected Host Cells and/or Expression Vectors

To determine the characteristics of host cells that have been selected by cell-sorting as high-performing host cells, or of expression constructs comprised by these host cells, DNA can be obtained from the sorted cells and used for analysis by DNA sequencing or for reconstruction of live host cells (see below) having genetic characteristics of high-performing host cells. For example, if the host cells comprise plasmid expression vectors, these can be recovered from selected high-performing host cells and sequenced by NGS. Genomic DNA can also be recovered from selected host cells and sequenced, but higher quantities of genomic DNA may be needed to achieve results comparable to those obtained from recovery of plasmid expression vectors. RNA can also be recovered from selected host cells, reverse-transcribed into DNA, and then analyzed by NGS and/or utilized by other methods.

Analysis of the recovered DNA by NGS can indicate which genetic attributes of the genetically diverse host cell population were enriched by the selection of high-performing host cells. For example, gene products that are coexpressed with the gene product of interest and that enhance expression levels of active gene product of interest can be identified from a large pool of coexpressed gene products. As another example, analysis of nucleic acids recovered from high-performing host cells can detect any genetic variation within the gene product of interest itself that is associated with an increased ability to bind to and/or act upon the labeling complex.

The fluorescence plots generated for a genetically diverse host cell population by FACS, representing the abilities of individual host cells to express a gene product of interest, preferably an active gene product of interest, can be divided into multiple different sectors. In some embodiments, a single sector is selected, using a cutoff to identify the cells having the highest fluorescence emissions. In some examples, the cutoff is the 0.05%-5% of cells, such as the top 0.05%-0.2%, 0.1-0.5%, 0.25-0.75%, 0.5-1%, 0.75-1.5%, 1%-2.5%, 2%-4%, or 3%-5% of cells, having the highest fluorescence emissions. In one example, the cutoff is the 0.5% of cells having the highest fluorescence emissions. However, one of ordinary skill in the art will recognize that higher or lower cutoff values may be used, depending on the capacity and type of cell sorting equipment being used. The cutoff is selected to provide uniformity between rounds of screening and/or between projects, and/or to reduce the amount of diversity in the enriched host cell population. In addition, the cutoff may depend on the number of cells sorted, such that a sufficient number of cells are included in the selected population of cells, for example, a sufficient number of cells to allow isolation of sufficient DNA for subsequent steps. Thus, in one non-limiting example, the cutoff is the 0.5% of cells having the highest fluorescence emissions and the minimum number of cells sorted is 200.

The host cells are sorted by FACS and the host cells corresponding to each sector are collected. NGS can then be used to determine the nucleotide sequences of the expression constructs in the host cells of various sectors, and in the unsorted genetically diverse population of host cells, preferably providing at least 10-fold, and more preferably at least 50-fold, repeated coverage of the unique sequences in the unsorted population and in each sorted sector.

The relative abundance of each unique sequence from the collected sectors is compared to the relative abundance of the unsorted host cell population. The fold change in relative abundance, computed by dividing the relative abundance of a unique sequence in the sorted host cells by the relative abundance of that sequence in the unsorted host cell population, is used to rank order each sequence, as a measure of its contribution to the expression of the gene product of interest. Nucleotide sequences that are enriched in sectors exhibiting high performance, and that are also depleted from sectors exhibiting low performance, are the best candidates for sequences that improve the expression of the gene product of interest.

It is also possible to ‘spike’ the host cell population with host cells from a characterized control strain, which comprise particular nucleotide sequences (“control nucleotide sequences”). These genetically homogeneous control host cells are likely to be sorted into one or a few sectors of the FACS plot, and NGS analysis of the control nucleotide sequences comprised by the control host cells should show that these sequences have the highest fold change in relative abundance in sorted host cells obtained from a few sectors of the FACS plot, identifying the level of fluorescence demonstrated by the control host cells. This optional ‘spiking’ procedure provides an internal benchmark for the fluorescence profile of the control host cell strain, which has been characterized for expression of the gene product of interest, allowing comparison of the fluorescence levels of the genetically diverse host cell population to that of the control host cells.

High-performing host cells that have been selected by cell-sorting methods such as FACS, for example the 0.1%, 1%, or 10% of the host cell population that displays the highest level of expression, can be characterized by a further FACS screening of the fluorescence or other detectable characteristic produced by the selected host cell population, to determine whether the cell-sorting and selection procedure has resulted in a population of host cells enriched for host cells with desirable properties. Further rounds of FACS sorting can be performed, with live or fixed cells as described above, to further enrich the host cell population for high-performing host cells.

When performing FACS sorting using live host cells, especially when multiple rounds of live-cell FACS sorting are to be employed, the selected populations of live host cells are typically cultured following the FACS procedure. To test for changes in the composition of a selected host cell population during culturing, a relatively small amount of the host cells (for example, 5-10% of the population) is removed prior to culturing, and reserved for NGS analysis. Another sample of host cells (20-50%, for example) can be removed following culturing (for a time consistent with one cell division, for example), for purposes such as determining the performance of the selected host cell population relative to a control host cell strain, as described above.

It can be advantageous to perform one or more initial rounds of FACS screening with live cells, as it is more effective to screen a highly genetically diverse host cell population with live cells that are less likely to form clumps of multiple cells. Once sufficient rounds of FACS selection with live cells have been performed, as shown by the proportion of the selected cells that have higher-performing characteristics when compared to a ‘spiked’ amount of control host cells, FACS can then be performed with fixed and labeled host cells to further enrich for host cells with the desired properties for production of active gene product of interest.

### V. Reconstructing Host Cell Strains

In certain examples, the expression constructs within the fixed host cells selected by cell sorting are harvested and sequenced by NGS. The sequences at each point of variation within the expression constructs are quantified, and those that are present at the greatest fold change in relative abundance, compared to the unsorted population. within the population are considered to be correlated with the high-performance characteristics of the selected host cells. However, sequencing by NGS obscures the linkage between points of variation on the expression vector, so it is not possible to determine whether the most prevalent sequence at position 2, for example, is usually associated with particular sequences at position 1 and position 3. A ‘high-performance’ library of expression vectors can be created including the most prevalent sequences at each point of variation, and creating the library of expression vectors to include all combinations of the prevalent sequences, including those that might display additive or synergistic properties created by particular combinations of sequences. This ‘high-performance’ library is then transformed into a parental strain of host cells, such as the E. coli strain 521 described above, to ‘reconstruct’ a population of live host cells having the genetic characteristics reflective of the selected high-performing host cells.

If the FACS scan of genetically diverse host cell population is compared to that of a ‘benchmark’ control host cell strain as described above, but the performance (as measured by FACS) of the genetically diverse population is not markedly higher than that of the control host cell strain, it can be advantageous to use NGS sequence data to create a ‘high performance” library as described above, to test for additivity or synergy between the highest-performing genetic sequences in the library in a further round of FACS screening. The creation of a ‘high performance” library can also be done after enrichment for high-performing host cells has been demonstrated, in order to determine if their performance can be further improved.

It is also possible to recover plasmid expression vectors from high-performing labeled and sorted host cells. The recovered plasmids can then be used to transform a parental host cell strain, and reconstruct a population of high-performing host cells.

Analysis of genomic DNA from selected high-performing host cells can also provide information about genetic characteristics that are associated with the desired high performance; these genetic characteristics can then be reintroduced into a parental host cell strain using the methods described under “Host Cell Population Genetic Diversity” in Section I.

### VI. Further Analyzing Reconstructed Host Cell Strains

Reconstructed host cells strains having genetic characteristics reflective of selected high-performing host cells can be analyzed by any method applicable to populations of cells expressing a gene product of interest. It can be useful to first isolate single host cells from a population of reconstructed host cells, by a FACS sort or by plating out host cells and picking and culturing individual colonies, in order to assess the performance of genetically homogeneous clonal populations derived from individual host cells.

Methods of determining which host cell populations or cultures exhibit the highest level of performance related to production of a gene product of interest can include quantifying isolated gene product(s) of interest by gel electrophoresis, enzyme-linked immunosorbent assay (ELISA), liquid chromatography (LC) including high-performance liquid chromatography (HP-LC), solid-phase extraction mass spectrometry (SPE-MS), and LC-MS (Example 1).

Methods to isolate gene product of interest from host cells, for the purpose of obtaining gene product of interest for further assessments of its quantity and activity, include high-throughput plate-based capture methods, such as those employing protein-A-based or KappaSelect (GE Healthcare Life Sciences, Marlborough, Mass.) solid media for the capture of antibodies.

For gene product(s) of interest that comprise disulfide bonds, the locations of these bonds within the gene product(s) can be determined by mass spectrometry as described in Example 1 below. Assays that determine the amount of active gene product(s) of interest can include antigen-binding assays, ligand-binding assays, enzymatic activity assays such as the cleavage of chromogenic substrates or chromogenic substrate analogs, and the binding of the gene product(s) of interest by antibodies specific for its active form. These types of assays can also be used to characterize variants of the gene product of interest that were identified in the host cell enrichment process, as a result of the variants' increased ability to bind and/or act upon the labeling complex used in the flow cytometry.

Host cells that exhibit the desired high-performance characteristics related to production of the gene product of interest can be grown in larger fermentation cultures to demonstrate the ability to produce the gene product of interest at scale, as described in Example 2.

### EXAMPLES

The following examples are provided to illustrate certain particular features and/or embodiments. These examples should not be construed to limit the disclosure to the particular features or embodiments described.

### Example 1

### Characterizing Disulfide Bonds

The number and location of disulfide bonds in polypeptide gene products can be determined by digestion of the polypeptide gene product with a protease, such as trypsin, under non-reducing conditions, and subjecting the resulting peptide fragments to mass spectrometry (MS) combining sequential electron transfer dissociation (ETD) and collision-induced dissociation (CID) MS steps (MS2, MS3) (Nili et al., “Defining the disulfide bonds of insulin-like growth factor-binding protein-5 by tandem mass spectrometry with electron transfer dissociation and collision-induced dissociation”, J Biol Chem 2012 Jan. 6; 287(2): 1510-1519; Epub 2011 Nov. 22).

Digestion of coexpressed protein. To prevent disulfide bond rearrangements, free cysteine residues are blocked by alkylation: the polypeptide gene product is incubated protected from light with the alkylating agent iodoacetamide (5 mM) with shaking for 30 minutes at 20 degrees C. in buffer with 4 M urea, and then is separated by non-reducing SDS-PAGE using precast gels. Alternatively, the polypeptide gene product is incubated in the gel after electrophoresis with iodoacetamide, or without as a control. Protein bands are stained, de-stained with double-deionized water, excised, and incubated twice in 0.5 mL of 50 mM ammonium bicarbonate, 50% (v/v) acetonitrile while shaking for 30 minutes at 20 degrees C. Protein samples are dehydrated in 100% acetonitrile for 2 minutes, dried by vacuum centrifugation, and rehydrated with 10 mg/ml of trypsin or chymotrypsin in buffer containing 50 mM ammonium bicarbonate and 5 mM calcium chloride for 15 minutes on ice. Excess buffer is removed and replaced with 0.05 mL of the same buffer without enzyme, followed by incubation for 16 hours at 37 degrees C. or at 20 degrees C., for trypsin and chymotrypsin, respectively, with shaking. Digestion is stopped by adding 3 microliters of 88% formic acid, and after brief vortexing, the supernatant is removed and stored at −20 degrees C. until analysis.

Localization of disulfide bonds by mass spectrometry. Peptides are injected onto a 1 mm×8 mm trap column (Michrom BioResources, Inc., Auburn, Calif.) at 20 microliters/minute in a mobile phase containing 0.1% formic acid. The trap cartridge is then placed in-line with a 0.5 mm×250 mm column containing 5 mm Zorbax SB-C18 stationary phase (Agilent Technologies, Santa Clara, Calif.), and peptides separated by a 2-30% acetonitrile gradient over 90 minutes at 10 micro-liters/minute with a 1100 series capillary HPLC (Agilent Technologies). Peptides are analyzed using a LTQ Velos linear ion trap with an ETD source (Thermo Scientific, San Jose, Calif.). Electrospray ionization is performed using a Captive Spray source (Michrom Bioresources, Inc.). Survey MS scans are followed by seven data-dependent scans consisting of CID and ETD MS2 scans on the most intense ion in the survey scan, followed by five MS3 CID scans on the first- to fifth-most intense ions in the ETD MS2 scan. CID scans use normalized collision energy of 35, and ETD scans use a 100 ms activation time with supplemental activation enabled. Minimum signals to initiate MS2 CID and ETD scans are 10,000, minimum signals for initiation of MS3 CID scans are 1000, and isolation widths for all MS2 and MS3 scans are 3.0 m/z. The dynamic exclusion feature of the software is enabled with a repeat count of 1, exclusion list size of 100, and exclusion duration of 30 s. Inclusion lists to target specific crosslinked species for collection of ETD MS2 scans are used. Separate data files for MS2 and MS3 scans are created by Bioworks 3.3 (Thermo Scientific) using ZSA charge state analysis. Matching of MS2 and MS3 scans to peptide sequences is performed by Sequest (V27, Rev 12, Thermo Scientific). The analysis is performed without enzyme specificity, a parent ion mass tolerance of 2.5, fragment mass tolerance of 1.0, and a variable mass of +16 for oxidized methionine residues. Results are then analyzed using the program Scaffold (V3_00_08, Proteome Software, Portland, Oreg.) with minimum peptide and protein probabilities of 95 and 99% being used. Peptides from MS3 results are sorted by scan number, and cysteine containing peptides are identified from groups of MS3 scans produced from the five most intense ions observed in ETD MS2 scans. The identities of cysteine peptides participating in disulfide-linked species are further confirmed by manual examination of the parent ion masses observed in the survey scan and the ETD MS2 scan.

### Example 2

### Fermentation

The fermentation processes involved in the production of gene products of interest can use a mode of operation which falls within one of the following categories: (1) discontinuous (batch process) operation, (2) continuous operation, and (3) semi-continuous (fed-batch) operation. A batch process is characterized by inoculation of the sterile culture medium (batch medium) with microorganisms at the start of the process, cultivated for a specific reaction period. During cultivation, cell concentrations, substrate concentrations (carbon source, nutrient salts, vitamins, etc.) and product concentrations change. Good mixing ensures that there are no significant local differences in composition or temperature of the reaction mixture. The reaction is non-stationary and cells are grown until the growth-limiting substrate (generally the carbon source) has been consumed.

Continuous operation is characterized in that fresh culture medium (feed medium) is added continuously to the fermenter and spent media and cells are drawn continuously from the fermenter at the same rate. In a continuous operation, growth rate is determined by the rate of medium addition, and the growth yield is determined by the concentration of the growth limiting substrate (i.e. carbon source). All reaction variables and control parameters remain constant in time and therefore a time-constant state is established in the fermenter followed by constant productivity and output.

Semi-continuous operation can be regarded as a combination of batch and continuous operation. The fermentation is started off as a batch process and when the growth-limiting substrate has been consumed, a continuous feed medium containing glucose and minerals is added in a specified manner (fed-batch). In other words, this operation employs both a batch medium and a feed medium to achieve cell growth and efficient production of the desired gene product(s). No cells are added or taken away during the cultivation period and therefore the fermenter operates batchwise as far as the microorganisms are concerned. While the present methods can be utilized in a variety of processes, including those mentioned above, a particular utilization is in conjunction with a fed-batch process.

In each of the above processes, cell growth and product accumulation can be monitored indirectly by taking advantage of a correlation between metabolite formation and some other variable, such as medium pH, optical density, color, and titratable acidity. For example, optical density provides an indication of the accumulation of insoluble cell particles and can be monitored on-stream using a micro-OD unit coupled to a display device or a recorder, or off-line by sampling. Optical density readings at 600 nanometers (OD600) are used as a means of determining dry cell weight.

High-cell-density fermentations are generally described as those processes which result in a yield of >30 g cell dry weight/liter (OD₆₀₀>60) at a minimum, and in certain embodiments result in a yield of >40 g cell dry weight/liter (OD₆₀₀>80). All high-cell-density fermentation processes employ a concentrated nutrient media that is gradually metered into the fermenter in a “fed-batch” process. A concentrated nutrient feed media is required for high-cell-density processes in order to minimize the dilution of the fermenter contents during feeding. A fed-batch process is required because it allows the operator to control the carbon source feeding, which is important because if the cells are exposed to concentrations of the carbon source high enough to generate high cell densities, the cells will produce so much of the inhibitory byproduct, acetate, that growth will stop (Majewski and Domach, “Simple constrained-optimization view of acetate overflow in E. coli”, Biotechnol Bioeng 1990 Mar. 25; 35(7): 732-738).

Acetic acid and its deprotonated ion, acetate, together represent one of the main inhibitory byproducts of bacterial growth in large-scale protein production in bioreactors. At pH 7, acetate is the most prevalent form of acetic acid. Any excess carbon energy source may be converted to acetic acid when the amount of the carbon energy source greatly exceeds the processing ability of the bacterium. Saturation of the tricarboxylic acid cycle and/or the electron transport chain is the most likely cause of the acetic acid accumulation. The choice of growth medium may affect the level of acetic acid inhibition; cells grown in defined media may be affected by acetic acid more than those grown in complex media. Replacement of glucose with glycerol may also greatly decrease the amount of acetic acid produced. It is believed that glycerol produces less acetic acid than glucose because its rate of transport into a cell is much slower than that of glucose. However, glycerol is more expensive than glucose, and may cause the bacteria to grow more slowly. The use of reduced growth temperatures can also decrease the speed of carbon source uptake and growth rate thus decreasing the production of acetic acid. Bacteria produce acetic acid not only in the presence of an excess carbon energy source or during fast growth, but also under anaerobic conditions. When bacteria such as E. coli are allowed to grow too fast, they may exceed the oxygen delivery ability of the bioreactor system which may lead to anaerobic growth conditions. To prevent this, a slower constant growth rate may be maintained through nutrient limitation. Other methods for reducing acetic acid accumulation include genetic modification to prevent acetic acid production, adding acetic acid utilization genes, and selection of strains with reduced acetic acid. E. coli BL21(DE3) is one of the strains that has been shown to produce lower levels of acetic acid because it can use acetic acid in its glyoxylate shunt pathway.

Larger-scale fed-batch fermenters are available for production of gene products of interest. Larger fermenters have at least 1000 liters of capacity, preferably about 1000 to 100,000 liters of capacity (i.e. working volume), leaving adequate room for headspace. These fermenters use agitator impellers or other suitable means to distribute oxygen and nutrients, especially glucose (the preferred carbon/energy source). Small-scale fermentation refers generally to fermentation in a fermenter that is no more than approximately 100 liters in volumetric capacity, and in some specific embodiments no more than approximately 10 liters.

Standard reaction conditions for the fermentation processes used to produce gene products of interest generally involve maintenance of pH at about 5.0 to 8.0 and cultivation temperatures ranging from 20 to 50 degrees C. for microbial host cells such as E. coli. In one embodiment, which utilizes E. coli as the host system, fermentation is performed at an optimal pH of about 7.0 and an optimal cultivation temperature of about 30 degrees C.

The standard nutrient media components in these fermentation processes generally include a source of energy, carbon, nitrogen, phosphorus, magnesium, and trace amounts of iron and calcium. In addition, the media may contain growth factors (such as vitamins and amino acids), inorganic salts, and any other precursors essential to product formation. The media may contain a transportable organophosphate such as a glycerophosphate, for example an alpha-glycerophosphate and/or a beta-glycerophosphate, and as a more specific example, glycerol-2-phosphate and/or glycerol-3-phosphate. The elemental composition of the host cell being cultivated can be used to calculate the proportion of each component required to support cell growth. The component concentrations will vary depending upon whether the process is a low-cell-density or a high-cell-density process. For example, the glucose concentrations in low-cell-density batch fermentation processes range from 1 to 5 g/L, while high-cell-density batch processes use glucose concentrations ranging from 45 g/L to 75 g/L. In addition, growth media may contain modest concentrations (for example, in the range of 0.1-5 mM, or 0.25 mM, 0.5 mM, 1 mM, 1.5 mM, or 2 mM) of protective osmolytes such as betaine, dimethylsulfoniopropionate, and/or choline.

One or more inducers can be introduced into the growth medium to induce expression of the gene product(s) of interest. Induction can be initiated during the exponential growth phase, for example, such as toward the end of the exponential growth phase but before the culture reaches maximum cell density, or at earlier or later times during fermentation. When expressing the gene product(s) of interest from one or more promoters inducible by depletion of nutrients such as phosphate, induction will occur when that nutrient has been sufficiently depleted from the growth medium, without the addition of an exogenous inducer.

During exponential growth of host cells, the metabolic rate is directly proportional to availability of oxygen and a carbon/energy source; thus, reducing the levels of available oxygen or carbon/energy sources, or both, will reduce metabolic rate. Manipulation of fermenter operating parameters, such as agitation rate or back pressure, or reducing O₂ pressure, modulates available oxygen levels and can reduce host cell metabolic rate. Reducing concentration or delivery rate, or both, of the carbon/energy source(s) has a similar effect. Furthermore, depending on the nature of the expression system, induction of expression can lead to a decrease in host cell metabolic rate. Finally, upon reaching maximum cell density, the growth rate stops or decreases dramatically. Reduction in host cell metabolic rate can result in more controlled expression of the gene product(s) of interest, including the processes of protein folding and assembly. Host cell metabolic rate can be assessed by measuring cell growth rates, either specific growth rates or instantaneous growth rates (by measuring optical density (OD) such as OD600 and or optionally by converting OD to biomass). The approximate biomass (cell dry weight) at each assayed point is calculated: approximate biomass (g)=(OD₆₀₀÷2)×volume (L). Desirable growth rates are, in certain embodiments, in the range of 0.01 to 0.7, or are in the range of 0.05 to 0.3, or are in the range of 0.1 to 0.2, or are approximately 0.15 (0.15 plus-or-minus 10%), or are 0.15.

Fermentation Equipment. The following are examples of equipment that can be used to grow host cells; many other configurations of fermentation systems are commercially available. Host cells can be grown in a New Brunswick BioFlo/CelliGen 115 water jacketed fermenter (Eppendorf North America, Hauppauge, N.Y.), 1 L vessel size with a 2× Rushton impeller and a BioFlo/CelliGen 115 Fermenter/Bioreactor controller; temperature, pH, and dissolved oxygen (DO) are monitored. It is also possible to grow host cells in a four-fold configurable DASGIP system (Eppendorf North America, Hauppauge, N.Y.) comprising four 60- to 250-ml DASbox fermentation vessels, each with a 2× Rushton impeller, a DASbox exhaust condenser, and a DASbox feeding and monitoring module (which includes a temperature sensor, a pH/redox sensor, and a dissolved oxygen sensor). Suitable fermentation equipment also includes NLF 22 30 L lab fermenters (Bioengineering, Inc., Somerville, Mass.), with 30-L capacity and 20-L maximum working volume in a stainless steel vessel; two Rushton impellers, sparged with air only; and a control system running BioSCADA software that allows for tracking and control of all relevant parameters including pH, DO, exhaust O₂, exhaust CO₂, temperature, and pressure.

### Example 3

### Activity-Specific Enrichment of Host Cells Expressing TRAST-Fab

TRAST-Fab is an antigen-binding fragment of the HER2-binding monoclonal antibody trastuzumab. The amino acid sequences of a TRAST-Fab heavy chain (‘HC’) and a TRAST-Fab light chain (‘LC’) are presented in SEQ ID NOs 2 and 3, respectively. In this Example, the heavy chain and the light chain of TRAST-Fab were coexpressed from an expression construct, the dual-promoter expression vector, which comprises an arabinose-inducible araBAD (‘ara’) promoter and a propionate-inducible prpBCDE (‘prp’) promoter. The nucleotide sequence of the dual-promoter expression vector is presented in SEQ ID NO:1. For the following activity-specific cell-enrichment procedures, the host cells were Escherichia coli 521 cells having the genotype shown in Section I above.

To create the populations of host cells for selection and activity-specific enrichment, E. coli 521 cells were transformed with the dual-promoter expression vector (SEQ ID NO:1), either without any additional polynucleotide sequences inserted into it (‘empty’, Sample A1 of Table 1), or comprising various polynucleotide sequences including those encoding TRAST-Fab, as described in Table 1 below. To allow an additional gene product to be expressed from the prp promoter, in certain samples the TRAST-Fab HC and LC were expressed in a bicistronic arrangement from the ara promoter, in either the HC-LC or the LC-HC arrangement. In some of those samples, the prp promoter expressed a polynucleotide encoding a form of the disulfide bond isomerase protein DsbC, which apparently lacks a signal peptide and thus is localized to the cell cytoplasm, and which will be referred to as ‘cDsbC’ (SEQ ID NO:4). The TRAST-Fab HC and LC polypeptides of SEQ ID NOs: 7 and 8 have an N-terminal amino acid sequence derived from Synechocystis sp. DnaB (UniProtKB Q55418); this DnaB-related amino acid sequence comprises a 6×His sequence and is provided as SEQ ID NO:9.

TABLE 1
Properties of Host Cell Populations for Activity-Specific Enrichment

| Sample | Expressed by | Expressed by | Other vector | Total |
|---|---|---|---|---|
| No. | ara promoter | prp promoter | diversity | diversity |
| A1 | — | — | — | 1 |
| ABS0258 | | | | |
| A2 | TRAST-Fab HC | TRAST-Fab LC | — | 1 |
| ABS0060 | (SEQ ID NO: 2) | (SEQ ID NO: 3) | | |
| A3 | bicistronic TRAST-Fab HC; LC | cDsbC (SEQ ID | — | 1 |
| ABS0804 | (SEQ ID NOs 5, 6) | NO: 4) | | |
| A4 | bicistronic TRAST-Fab DnaB-HC; | cDsbC (SEQ ID | — | 1 |
| ABS0861 | DnaB-LC (SEQ ID NOs 7, 8) | NO: 4) | | |
| B1 | bicistronic TRAST-Fab DnaB-HC; | 137 different | ×12,769 | 1,749,353 |
| ABS1411 | DnaB-LC (SEQ ID NOs 7, 8) | gene products | | |
| B2 | bicistronic TRAST-Fab DnaB-LC; | 137 different | ×12,769 | 1,749,353 |
| ABS1412 | DnaB-HC (SEQ ID NOs 8, 7) | gene products | | |
| B3 | bicistronic TRAST-Fab DnaB-HC; | 137 different | ×144 | 19,728 |
| ABS1413 | DnaB-LC (SEQ ID NOs 7, 8) | gene products | | |
| B4 | bicistronic TRAST-Fab DnaB-LC; | 137 different | ×144 | 19,728 |
| ABS1414 | DnaB-HC (SEQ ID NOs 8, 7) | gene products | | |
| B5 | bicistronic TRAST-Fab DnaB-HC; | cDsbC (SEQ ID | ×12,769 | 12,769 |
| ABS1415 | DnaB-LC (SEQ ID NOs 7, 8) | NO: 4) | | |
| B6 | bicistronic TRAST-Fab DnaB-LC; | cDsbC (SEQ ID | ×12,769 | 12,769 |
| ABS1416 | DnaB-HC (SEQ ID NOs 8, 7) | NO: 4) | | |
| C1 | bicistronic TRAST-Fab DnaB-HC; | 137 different | ×12,769 | 1,749,353 |
| ABS1411 | DnaB-LC (SEQ ID NOs 7, 8) | gene products | | |
| C2 | bicistronic TRAST-Fab DnaB-LC; | 137 different | ×12,769 | 1,749,353 |
| ABS1412 | DnaB-HC (SEQ ID NOs 8, 7) | gene products | | |
| C3 | bicistronic TRAST-Fab DnaB-HC; | 137 different | ×144 | 19,728 |
| ABS1413 | DnaB-LC (SEQ ID NOs 7, 8) | gene products | | |
| C4 | bicistronic TRAST-Fab DnaB-LC; | 137 different | ×144 | 19,728 |
| ABS1414 | DnaB-HC (SEQ ID NOs 8, 7) | gene products | | |

Samples A1-A4 were control samples for the procedure, A1 being a negative control host cell population expressing no TRAST-Fab gene product, and A2-A4 being control host cell populations that each express TRAST-Fab from a single form of the expression vector. In samples B1-B4 and C1-C4, the host cell populations comprised diverse forms of the expression vector with 137 different gene products expressed from the prp promoter. In samples B1-B6 and C1-C4, the expression vectors comprised by the host cells had further sources of variation that increased the total number of different forms of the expression vector within the population to 12,769, 19,728, or 1,749,353.

Following transformation with the expression vector(s), the host cell samples were plated onto solid media containing kanamycin (50 micrograms/mL) to select for successful transformants comprising expression vectors, which carry a gene for kanamycin resistance. After growth at 37 degrees C. overnight, the host cell colonies were scraped off the solid media into LB medium (10 g/L tryptone, 5 g/L yeast extract, and 10 g/L NaCl), and the optical density at 600 nm (OD600) was adjusted by dilution with LB medium to 3. The host cell populations were induced for expression of TRAST-Fab HC and LC, and any other gene products present on the expression vector, in induction medium (fermentation production medium with 8 mM MgSO4, 1× Korz trace metals, 50 micrograms/mL kanamycin, and inducers as described below).

The fermentation production medium included KH2PO4, (NH4)2SO4, yeast extract, glycerol, citric acid, and 1× Korz trace metals, with NH4OH to bring to pH 6.8.

Samples A1, A3, A4, and B1-B6 were induced in media containing 1 mM propionate and 250 micromolar arabinose; samples A2 and C1-C4 were induced in media containing 20 mM propionate and 250 micromolar arabinose.

Two duplicates of each host cell population (at an OD600 of 3, above) were placed into induction medium in a 24-well deep-well plate, covered with an Aeraseal™ plate cover (Excel Scientific, Victorville, Calif.), incubated, and then the OD600 of each sample determined. The remaining host cell culture in each sample was harvested for further analysis by centrifuging, followed by aspiration of the supernatants and stored as pellets.

The samples were then fixed for labeling. The host cells were fixed by adding 0.5 mL of cold fixation solution (0.65% paraformaldehyde, 0.02% glutaraldehyde, and 32.25 mM tribasic sodium phosphate in deionized water) to each sample and resuspending the pellet, incubating, centrifuging, and removing the supernatant by aspiration. A 0.2-mL volume of permeabilization buffer (50 mM glucose, 20 mM Tris, 10 mM EDTA pH 8.2, and 1 unit of lysozyme per 10 mL of buffer in deionized water) was added to each washed pellet, and the samples were incubated on ice. Following incubation in permeabilization buffer, the samples were centrifuged while cold, and the supernatant removed by aspiration. The permeabilized host cell pellets were fixed by adding 0.5 mL 1× Immunoassay Buffer (PerkinElmer, Waltham Mass., 25 mM HEPES pH 7.4, 0.1% Casein, 1 mg/mL Dextran-500, 0.5% Triton X-100 and 0.05% Proclin-300, plus 1 mM EDTA) to the pellets without mixing, the samples were centrifuged, and the supernatant removed by aspiration.

To label the TRAST-Fab within the permeabilized and fixed host cells, the HER2 antigen, which is specifically bound by the TRAST-Fab antibody fragment, was first conjugated to biotin in the presence of fluorescently labeled streptavidin, to prepare a HER2-biotin-streptavidin-fluorophore conjugate. A mixture of 10 micromolar Alexa Fluor® 488 streptavidin (ThermoFisher Scientific Inc., Waltham, Mass.) and 1.75 micromolar HER2 (about 1:20 v/v) was brought up to 10 mL by addition Immunoassay Buffer (see above) with 1 mM EDTA. The tube containing this solution incubated overnight at 4 degrees C. on a rotating mixer. After the incubation, biotin was added to the HER2 Alexa Fluor® 488 streptavidin solution (0.1 mg/mL biotin final concentration) and was incubated.

The host cell samples were labeled by addition of the HER2-biotin-streptavidin-Alexa Fluor® 488 solution to each sample and incubated overnight at 4 degrees C. The samples were then centrifuged, and the supernatant was removed by aspiration. The host cell pellets were resuspended in 0.5 mL 1×PBS pH 8 for the FACS selection procedure.

A FACS instrument, BD FACSAria™-IIu (Becton, Dickinson and Co., Franklin Lakes, N.J.) was used for sorting of the labeled host cells in the samples. Propidium iodide (1 mg/ml) was added to each 0.5-mL sample to stain the DNA present in the host cells. Because the host cells in the samples were fixed and permeabilized, the propidium iodide was able to penetrate the host cells and access the cells' DNA. The host cell samples, A1-A4, B1-B6, and C1-C4 as shown in Table 1, were run without sorting on the FACS instrument to set up the voltages for the photomultiplier tubes (PMTs) being used in the experiment. The host cell samples were run through the FACS instrument, 50,000 events for each A1-A4 control sample and 1 million events for each of the B1-B6 and C1-C4 samples were recorded, with duplicate runs for each sample except for A4. Based on the experimental data generated from the samples, sorting gates were set up using FlowJo™ software (Becton, Dickinson and Co., Franklin Lakes, N.J.) that determined the parameters at which sorting of the labeled host cells will occur.

The first gating criterion was based on DNA fluorescence detection, using a 675/20 nm wavelength filter, plotted as SSC-A (total cell granularity) against FSC-A (total cell fluorescence as an indicator of cell size). For the fixed and labeled E. coli host cells used in this experiment, increases in size and granularity are likely to arise from clumping of multiple cells. This initial gate (‘P2’) was set to retain over 99.9% of the detection events interrogated and to exclude only those events that were extreme outliers when compared to the expected SSC-A to FSC-A distribution. The second gate was also based on 675/20 DNA fluorescence, plotted as SSC-W against FSC-H, and the selected events were set to be those with a SSC-W value between 38,000 and 63,000—the range expected for a single cell—to eliminate clumps of multiple cells, and an FSC-H value of 20 or greater. The second gate resulted in the retention of between approximately 30% and 50% of the detection events, depending on the sample.

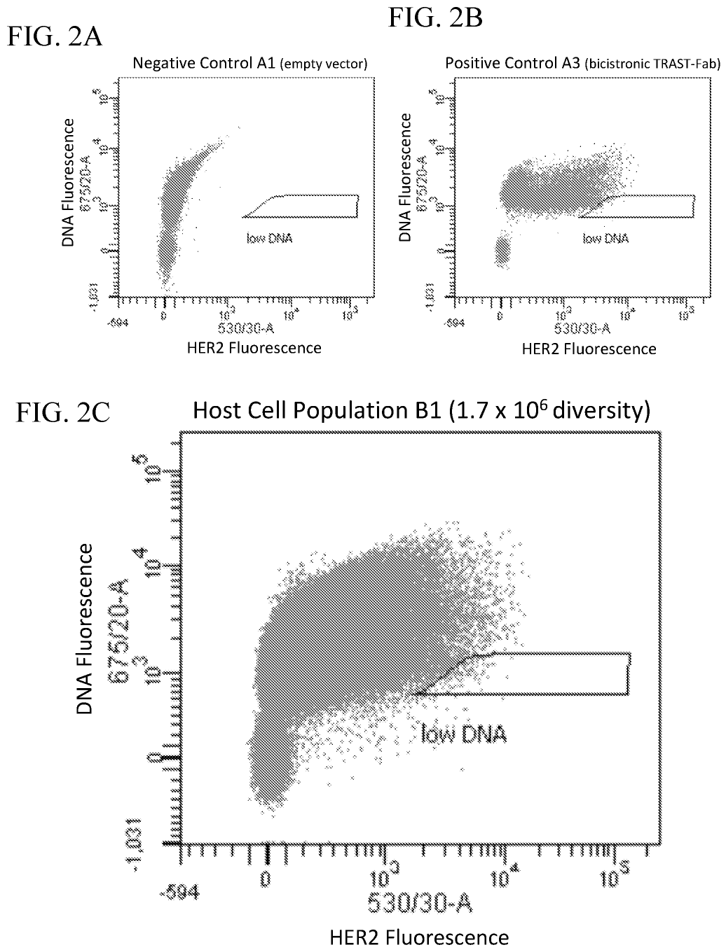

The final sorting gate was based on a comparison of 675/20 DNA fluorescence, measured as FSC-A, plotted against 530/30 fluorescence of the HER2-labeled TRAST-Fab protein or DnaB-TRAST-Fab, measured as FSC-A. A ‘low DNA’ gate was created with complex boundaries, as shown in FIG. 2. This gate selected detection events associated with lower amounts of DNA fluorescence, and higher amounts of HER2-labeled Alexa Fluor® 488 fluorescence, to select individual cells with higher production of TRAST-Fab or DnaB-TRAST-Fab. When this ‘low DNA’ gate was applied to the 50,000 events recorded for the control samples A1-A4, zero events were selected with this gate for the A1 ‘empty vector’ control sample, 1 event for the A4 control sample, and 2434 and 1471 events (the averages of the two runs) for samples A2 and A3, respectively. Application of the ‘low DNA’ gate to the one million events recorded for the B1-B6 and C1-C4 samples resulted in an average for each sample of between 82 and 662 events being selected.

Prior to starting the cell-sorting operation, 50 microliters of 1× Immunoassay Buffer (see above), without EDTA, was placed into the collection tubes. The cell sorting was performed on samples B1-B6 and C1-C4, with between 2.7 million and 10.9 million events recorded and 1000 events collected per sample.

The FACS-sorted samples comprising host cells that exhibit high levels of DnaB-TRAST-Fab expression were prepared for further analysis by isolating plasmid DNA from the selected cell populations using a QIAprep® Spin Miniprep Kit (Qiagen, Venlo, Netherlands) according to the manufacturer's instructions, for the purpose of reconstructing host cells (below), and for high-throughput next-generation DNA sequencing (‘NGS’). Also prepared for NGS analysis were the corresponding pre-sort samples. The DNA samples for NGS were prepared by mixture with Nextera Flex beads (Illumina, San Diego, Calif.). The ‘tagmented’ DNA samples were then amplified by polymerase chain reaction (PCR) and run on a MiSeq sequencer (Illumina, San Diego, Calif.).

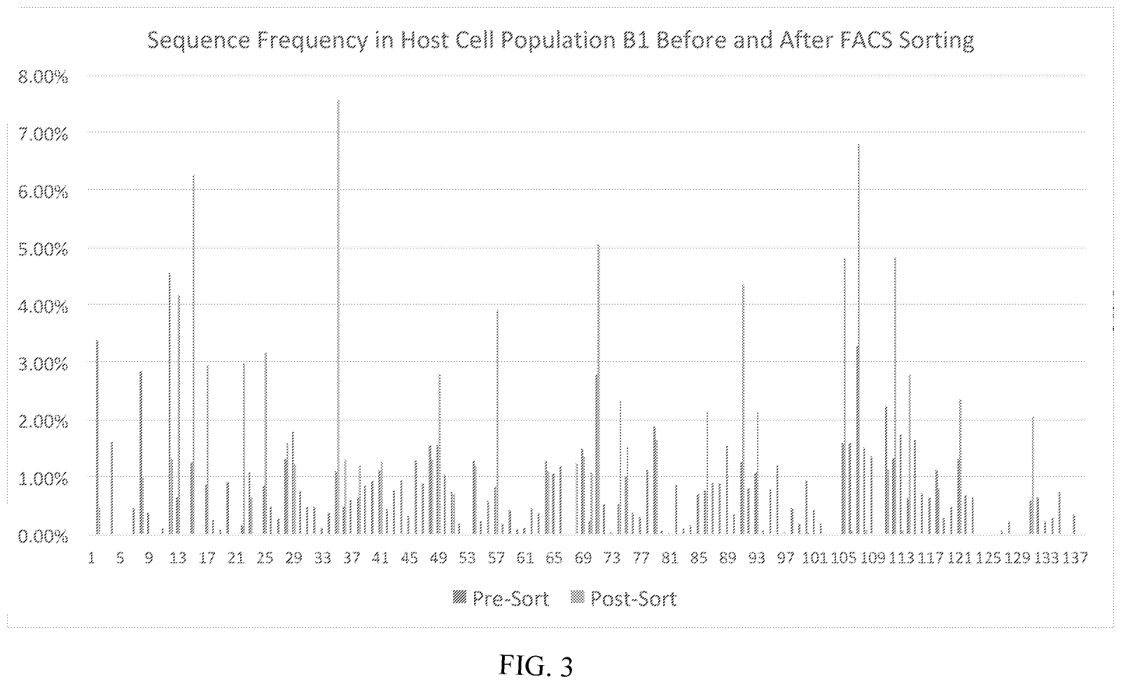

When compared to the corresponding pre-sort samples, the populations of host cells selected for higher DnaB-TRAST-Fab expression by FACS sorting were found by NGS to be enriched for the presence of particular expression vector polynucleotide elements and for certain gene products coexpressed with DnaB-TRAST-Fab from the prp promoter, as shown in FIG. 3.

The plasmid DNA recovered from the high-expressing host cells was also used to transform the parental host cell strain, E. coli 521 cells, to reconstruct host cell populations enriched for expression vectors that direct high levels of expression of DnaB-TRAST-Fab.

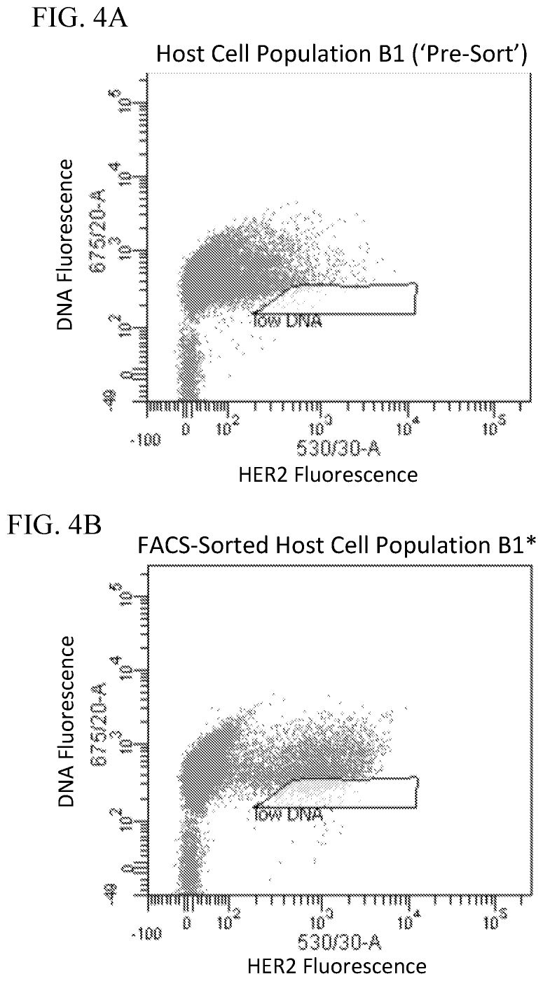

The reconstructed host cell populations, corresponding to the host cells selected from samples B1-B6 and C1-C4 in Table 1 above, were referred to as B1*-B6* and C1*-C4* to indicate that they were reconstructed from FACS-selected host cells. These B1*-B6* and C1*-C4* host cell populations, along with previously unsorted host cell populations B1-B6 and C1-C4 as described in Table 1, were grown, induced by incubation in induction medium for 22 hours, harvested, labeled, and analyzed by a gated FACS screen as described above. The B1*-B6* and C1*-C4* populations of host cells that resulted from FACS sorting were significantly enriched for host cells that express TRAST-Fab at a higher level, as shown in FIG. 4.

The FACS-selected B1*-B4* host cell populations were reconstructed as described in Example 1E above: the plasmids recovered from each sample were transformed into the E. coli 521 parental host cell strain and plated out on solid media containing 50 micrograms/mL kanamycin. Individual colonies of host cells were picked into 96-well plates—88 wells for B1*, 163 wells for B2*, 88 wells for B3*, and 189 wells for B4*—in order to determine the expression of TRAST-Fab by host cell cultures derived from individual cells. Control host cells A3 and A4 (see Table 1) were also included in multiple wells on each 96-well plate. These host cell samples were grown and TRAST-Fab expression was induced by incubation in induction medium, generally using the procedures set out in Example 1A. A predetermined volume (200 microliters) was removed from each induced host cell culture into a fresh 96-well plate for the purpose of determining the TRAST-Fab expression levels of these aliquoted samples by SPE-MS.

The harvested host cell samples (A3, A4, and B1*-B4*) were lysed, and the samples were centrifuged. Each sample was transferred into digestion buffer (8 M urea, 200 mM histidine at pH 6.00, 1:1 v/v), then heated to aid in unfolding the proteins. Following heating, trypsin/lysC protease mixture (Promega, Madison Wis.) was added to each well. The samples were then incubated. Following incubation the samples were quenched with the addition of formic acid.

The digested and quenched samples of host cell proteins from samples A3, A4, and B1*-B4* were then subjected to SPE-MS for peptide multiple reaction monitoring (MRM) detection. The MRM was set up to monitor three peptides from the DnaB-TRAST-Fab polypeptides of the samples: a peptide from the heavy chain (HC), GPSVFPLAPSSK (amino acids 126-137 of SEQ ID NO:2); a peptide from the light chain (LC), DSTYSLSSTLTLSK (amino acids 171-184 of SEQ ID NO:3); and a peptide from the DnaB-related N-terminal amino acid sequence, EHIALPR (amino acids 92-98 of SEQ ID NO:9). These peptides were chosen to provide optimal declustering potential and collision energies. Based on these criteria, two transitions per peptide were monitored, as shown below in Table 2.

TABLE 2
Descriptive characteristics of the DnaB-TRAST-Fab
MRM experiment.

| | Peptide Amino | | | Transi- | Transi- |
|---|---|---|---|---|---|
| Peptide | Acid (AA) | | Q1 | tion | tion |
| Source | Sequence | Charge | Mass | 1 | 2 |
| TRAST_ | GPSVFPLAPSSK | 2 | 594.08 | 418.4 | 700.4 |
| HC | SEQ ID NO: 2 | | | | |
| | AAs 126-137 | | | | |
| TRAST_ | DSTYSLSSTLTLSK | 2 | 752.21 | 836.5 | 1037.8 |
| LC | SEQ ID NO: 3 | | | | |
| | AAs 171-184 | | | | |
| DnaB | EHIALPR | 2 | 418.0 | 569.5 | 456.0 |
| | SEQ ID NO: 9 | | | | |
| | AAs 92-98 | | | | |

TRAST-Fab standard was digested in series of dilution samples prepared by diluting the standard with cell lysate prepared from ‘empty’ (no expression vector) host cells. The standard curve generated by this procedure was used for quantification of all interrogated samples.

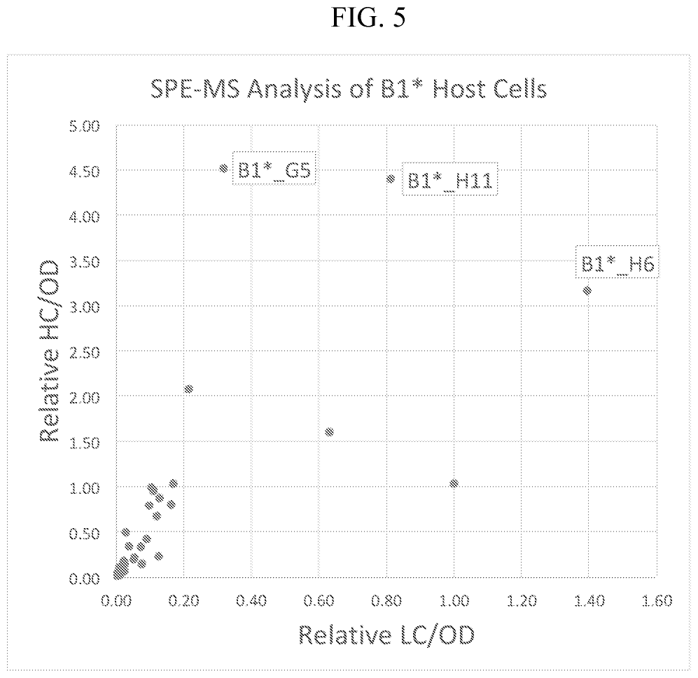

Candidate host cell populations were selected based on expressing high amounts of both HC and LC (mg/L/OD600), relative to the A3 control sample shown in Table 1, and also on exhibiting at least 2.5 times higher levels of DnaB intein, corresponding to higher total protein production, than the control sample A3 (see FIG. 5). Samples B1*_G5, B1*_H11, B1*_H6, B2*_A10, and B4*_H11 were selected for further analysis by protein-A-based purification and by an antigen-binding assay for functional TRAST-Fab, as described further below.

The host cells from samples B1*_G5, B1*_H11, B1*_H6, B2*_A10, and B4*_H11 and control sample A3 were grown in 20 mL of shake flask culture generally as described in Example 1A, the OD600 of each culture was measured, and then they were centrifuged to form pellets of host cells. The host cells were lysed, and incubation on ice for 30 minutes. The host cell lysates were centrifuged, and the supernatant was filtered. Filtered cell lysate was loaded onto an ÄKTA™ HiTrap MabSelect™ 1-mL column (GE Healthcare Life Sciences, Marlborough, Mass.) for protein-A-based purification of the TRAST-Fab or DnaB-TRAST-Fab heavy chain/light chain heterodimer (collectively referred to as TRAST-Fab heterodimer) in the host cell lysates from the samples, through binding of the Fab heavy chain by protein A. The ÄKTA™ device measured the absorbance of the eluate fractions at 280 nm, and integrated the results for each sample to determine the total amount of protein present in the eluate peak. In addition, HP-LC was used to quantify the amount of TRAST-Fab heterodimer present in the eluate fractions, based on the 280 nm absorbance peak corresponding to the expected mass of the heterodimer. The results are shown in Table 3 below, where the amount of protein is expressed in terms of the volume and cell density (OD600) of the induced host cell culture. From this analysis it can be seen that the B4*_H11 sample consistently produced about 1.5 times as much total protein and TRAST-Fab heterodimer as the control A3 sample.

TABLE 3
Quantification of TRAST-Fab Production by Individual Host Cells

| | | Total | TRAST-Fab |
|---|---|---|---|
| | | Protein-A-Binding | Heterodimer |
| Sample | OD600 | (mg/L/OD600) | (mg/L/OD600) |
| A3 Control | 19.56 | 14.62 | 7.07 |
| B1*_G5 | 16.84 | 8.06 | 11.15 |
| B1*_H11 | 17.08 | 10.05 | 3.81 |
| B1*_H6 | 17.56 | 25.49 | 5.02 |
| B2*_A10 | 20.36 | 22.10 | 2.95 |
| B4*_H11 | 16.64 | 20.82 | 11.20 |

The amount of active DnaB-TRAST-Fab produced by samples B1*_G5, B1*_H11, B1*_H6, B2*_A10, and B4*_H11, and of active TRAST-Fab by control sample A3, was assessed by an antigen-binding assay that specifically measures the presence of TRAST-Fab heterodimer having antigen-binding activity. This assay indicated that samples B2*_A10 and B4*_H11 each produced about 1.5 times as much TRAST-Fab heterodimer as the control A3 sample.

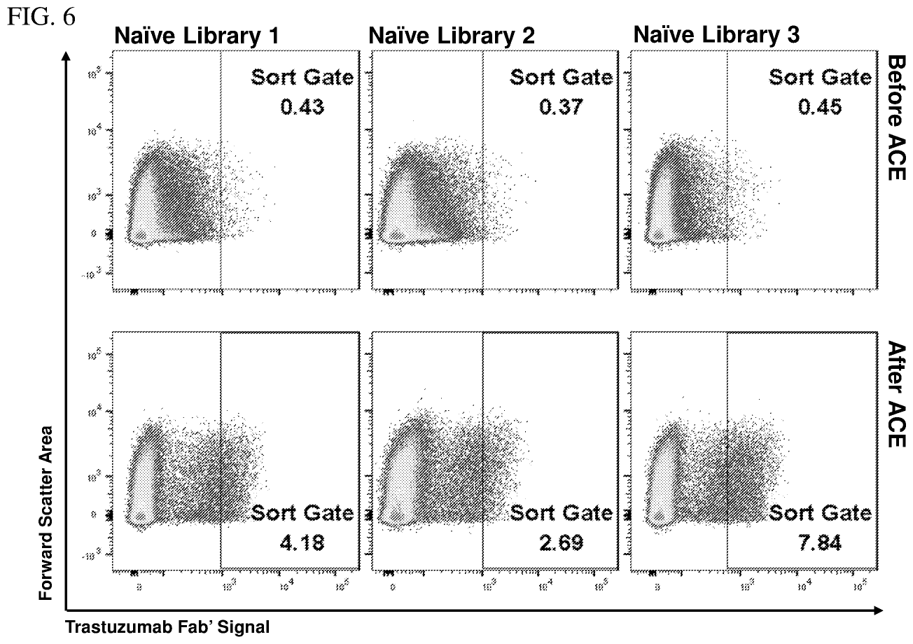

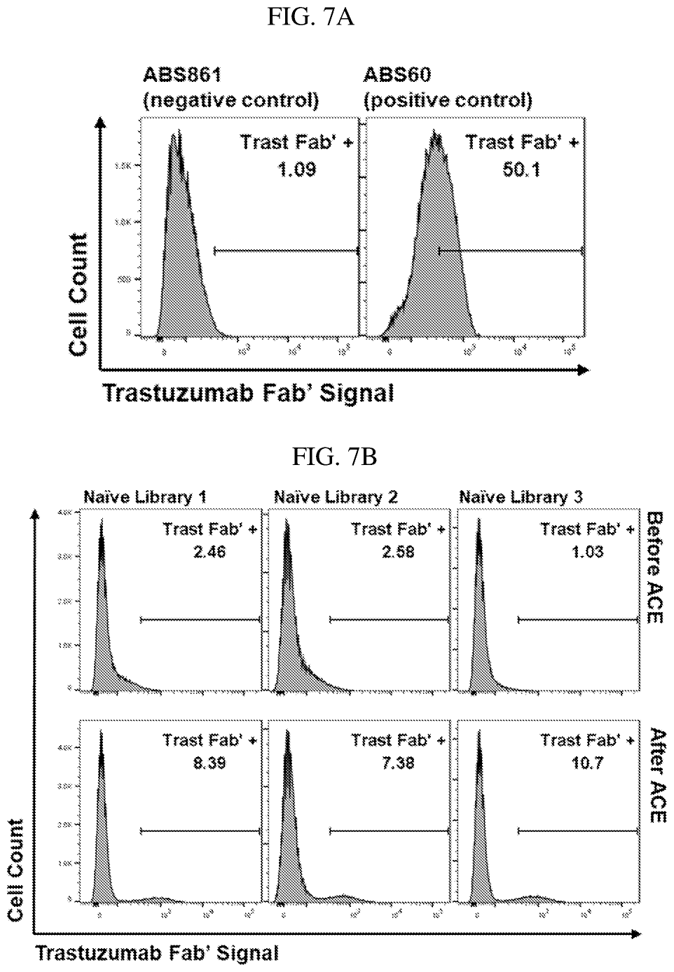

The level of enrichment of high-expressing vectors was evaluated. Using the ACE assay, naïve libraries were fixed, permeabilized, and probed with HER2 to detect the production of the Trastuzumab Fab′. Of target producing cells, the top <0.5% were sorted via ACE assay. Subsequently, vector plasmid was isolated and re-transformed into cells to assess expression. Applying the same sort gate, after re-transformation demonstrates >10-fold enrichment for high-expressing vectors (FIG. 6). To assess the full increase in Trastuzumab Fab′ production after ACE assay, gating was established by negative and positive control samples. Naive libraries typically had low level expression that was greatly increased after ACE assay (FIGS. 7A-7B).

In practicing the present disclosure, many conventional techniques in molecular biology, microbiology, and recombinant DNA technology are optionally used. Such conventional techniques relate to vectors, host cells, and recombinant methods. These techniques are well known and are explained in, for example, Berger and Kimmel, Guide to Molecular Cloning Techniques, Methods in Enzymology volume 152 Academic Press, Mc, San Diego, Calif.; Sambrook et al., Molecular Cloning—A Laboratory Manual (3rd Ed.), Vol. 1-3, Cold Spring Harbor Laboratory, Cold Spring Harbor, N.Y., 2000; and Current Protocols in Molecular Biology, F. M. Ausubel et al., eds., Current Protocols, a joint venture between Greene Publishing Associates, Inc. and John Wiley & Sons, Inc., (supplemented through 2006). Other useful references, for example for cell isolation and culture and for subsequent nucleic acid or protein isolation, include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York); and Atlas and Parks (Eds.) The Handbook of Microbiological Media (1993) CRC Press, Boca Raton, Fla. Methods of making nucleic acids (for example, by in vitro amplification, purification from cells, or chemical synthesis), methods for manipulating nucleic acids (for example, by site-directed mutagenesis, restriction enzyme digestion, ligation, etc.), and various vectors, cell lines, and the like useful in manipulating and making nucleic acids are described in the above references. In addition, essentially any polynucleotide (including labeled or biotinylated polynucleotides) can be custom or standard ordered from any of a variety of commercial sources.

The present invention has been described in terms of particular embodiments found or proposed to comprise certain modes for the practice of the invention. It will be appreciated by those of ordinary skill in the art that, in light of the present disclosure, numerous modifications and changes can be made in the particular embodiments exemplified without departing from the intended scope of the invention.

All cited references, including patent publications, are incorporated herein by reference in their entirety. Nucleotide and other genetic sequences, referred to by published genomic location or other description, are also expressly incorporated herein by reference.

We claim:

1. A method for selecting host cells from a population of host cells having genetic diversity of at least 1000, wherein at least some of the host cells comprise a polynucleotide sequence encoding a gene product of interest, the method comprising:
culturing the population of host cells, whereby the gene product of interest is expressed by a subpopulation of the host cells of the population, the subpopulation thereby comprising expressing host cells;
labeling at least some of the expressing host cells of the subpopulation, wherein the labeling comprises associating the gene product of interest with a detectable moiety, thereby producing labeled expressing host cells; and
selecting a subset of labeled expressing host cells, wherein the selecting comprises detecting the detectable moiety by a cell-sorting apparatus.

2. The method of claim 1, wherein the genetic diversity of the host cell population is host cell genomic variation, polynucleotide sequence variation of one or more expression constructs, or a combination thereof, comprised by at least some of the host cells of the host cell population.

3. The method of claim 2, wherein the genetic diversity of the population of host cells is 200,000-1,000,000.

4. A method for selecting expressing host cells from a population of host cells having a genetic diversity, the genetic diversity comprising a plurality of genetic variants, wherein at least some of the host cells comprise a polynucleotide sequence encoding a gene product of interest, the method comprising:
culturing the population of host cells, whereby the gene product of interest is expressed by a subpopulation of the host cells of the population, the subpopulation thereby comprising expressing host cells, wherein levels of the expression of the gene product of interest from the expressing host cells varies based on the genetic variant;
labeling at least some of the expressing host cells of the subpopulation, wherein the labeling comprises associating the gene product of interest with a detectable moiety, wherein an amount of the labeling is proportional to the expression level of the gene product of interest in the expressing host cell, thereby producing labeled expressing host cells; and
selecting a subset of labeled expressing host cells, wherein the selecting comprises detecting the detectable moiety and the amount of labeling by a cell-sorting apparatus.

5. A method for selecting expressing host cells from a population of host cells having a genetic diversity, the genetic diversity comprising a plurality of genetic variants, wherein at least some of the host cells comprise a polynucleotide sequence encoding a gene product of interest, the method comprising:
culturing the population of host cells, whereby the gene product of interest is expressed by a subpopulation of the host cells of the population, the subpopulation thereby comprising expressing host cells, wherein a predetermined property of the expressing host cells varies based on the genetic variant;
labeling at least some of the expressing host cells of the subpopulation, wherein the labeling comprises associating the gene product of interest with a detectable moiety, wherein an amount of the labeling proportional to the predetermined property of the gene product of interest in the expressing host cell, thereby producing labeled expressing host cells; and
selecting a subset of labeled expressing host cells, wherein the selecting comprises detecting the detectable moiety and the predetermined by a cell-sorting apparatus.

6. The method of claim 5, wherein the predetermined property of the expressing host cells comprises level of expression of active gene product of interest, level of expression of the gene product of interest, proper protein folding of the gene product of interest, level of expression of properly folded protein of the gene product of interest, cell viability, and/or amount of biomass.

7. The method of any one of claims 1 to 5, further comprising measuring relative expression level of the gene product of interest for each genetic variant

8. The method of any one of claims 1 to 5, wherein the selecting comprises fluorescence-activated cell sorting.

9. The method of any one of claims 1 to 5, wherein the detectable moiety comprises a fluorescent moiety and the selecting comprises selecting the 0.01%-5% of cells with highest fluorescence emissions.

10. The method of claim 9, wherein the selecting comprises selecting the 0.5% of cells with highest fluorescence emissions

11. The method of any one of claims 1 to 5, wherein the gene product of interest comprises a polypeptide lacking a signal peptide.

12. The method of any one of claims 1 to 5, wherein the gene product of interest comprises a first polypeptide fused in-frame to a second polypeptide selected from the group consisting of a fluorescent polypeptide and a bioluminescent polypeptide.

13. The method of claim 12, wherein the detectable moiety associated with the gene product of interest comprises the polypeptide selected from the group consisting of a fluorescent polypeptide and a bioluminescent polypeptide.

14. The method of any one of claims 1 to 5, wherein the gene product of interest comprises a first polypeptide fused in-frame to a second polypeptide having enzymatic activity.

15. The method of claim 14, wherein the detectable moiety associated with the gene product of interest is bound to the active site of the polypeptide having enzymatic activity.

16. The method of any one of claims 1 to 5, wherein the polynucleotide sequence encoding the gene product of interest is an expression vector.

17. The method of claim 16, wherein the expression vector is an extrachromosomal expression vector.

18. The method of any one of claims 1 to 5, wherein labeling at least some of the expressing host cells of the subpopulation comprises fixing the subpopulation of expressing host cells.

19. The method of claim 18, wherein fixing the subpopulation of expressing host cells comprises contacting at least some of the expressing host cells of the subpopulation with an aldehyde.

20. The method of claim 19, wherein the aldehyde is paraformaldehyde.

21. The method of any one of claims 1 to 5, wherein labeling at least some of the expressing host cells of the subpopulation comprises permeabilizing at least some of the expressing host cells of the subpopulation.

22. The method of claim 21, wherein permeabilizing at least some of the expressing host cells of the subpopulation comprises contacting at least some of the expressing host cells of the subpopulation with lysozyme.

23. The method of any one of claims 1 to 5, wherein labeling at least some of the expressing host cells of the subpopulation further comprises contacting at least some of the expressing host cells of the subpopulation with a compound that labels DNA.

24. The method of claim 23, wherein the compound that labels DNA is propidium iodide.

25. The method of any one of claims 1 to 5, wherein the host cells of the population of host cells are prokaryotic cells.

26. The method of claim 25, wherein the host cells of the population of host cells are Escherichia coli cells.

27. The method of claim 26, wherein the host cells of the population of host cells are Escherichia coli 521 cells.

28. The method of any one of claims 1 to 5, further comprising the recovery of polynucleotides from the subset of labeled expressing host cells, thereby producing recovered polynucleotides.

29. The method of claim 28, further comprising obtaining DNA sequence information from the recovered polynucleotides.

30. The method of claim 29, further comprising modifying the genome of a host cell based upon the DNA sequence information.

31. The method of claim 30, further comprising constructing a library of expression vectors based upon the DNA sequence information.

32. The method of claim 31, further comprising transforming a parental host cell strain with the library of expression vectors.

33. The method of claim 28, wherein the recovered polynucleotides are expression vectors.

34. The method of claim 33, further comprising transforming a parental host cell strain with one or more of the expression vectors.

35. The method of claim 32 or claim 34, further comprising culturing the transformed host cells.

36. The method of claim 35, wherein at least some of the transformed host cells express the gene product of interest.

37. The method of claim 36, further comprising determining the level of expression of the gene product of interest by a method selected from the group consisting of gel electrophoresis, enzyme-linked immunosorbent assay (ELISA), liquid chromatography (LC) including high-performance liquid chromatography (HP-LC), solid-phase extraction mass spectrometry (SPE-MS), and an Amplified Luminescent Proximity Homogeneous Assay.


SEQUENCE LISTING (9 sequences)

SEQ ID NO: 1 - DNA, 5304 bp, Artificial Sequence
  Expression vector
ggcctttcttcggtagaagtcttcccccagaggcaggtatcaaaggatcttcttgagatcctttttttctgcgcgtaatctgctgcttgcaaacaaaaaaaccaccgctaccagcggtggtttgtttgccggatcaagagctaccaactctttttccgaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcatcgatttttgtgatgctcgtcaggggggcggagcctatggaaaaacgccagcaacgcagaaaggcccacccgaaggtgagccaggtgattacatttgggccctcatcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcatacctgcttagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatagctcctgaaaatctcgataactcaaaaaatacgcccggtagtgatcttatttcattatggtgaaagttggaacctcttacgtgccgatcaagaagacggtcaaaagcctccggtcggaggccgggagagtgttcaccgacaaacaacagataaaacaaaaggcccagtcttccgactgagccttttgttttatttgatgtctggcagttcccgagacgttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtctctgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcaggaggtaaaaaaaatgtgagaccggtctcggtctagatcggtcagtttcacctgatttacgtaaaaacccgcttcggcgggtttttgcttttggaggggcagaaagatgaatgactgtctctcctgttagtgagggttaatgcccggaacgaagaaaggcccacccgtgaaggtgagccagtgagttggttacattttctcttgagggtttagcttttcagacgacgccaaaaggtcgtacgtgaaatacccaaatagttggccgcagccgtcttgtcaccattaaacttctcaagcgcttgctgcggggtcagcaaacgcggagccggcgtctttgcgctctcacgcgccagctccggcagcagcaactgcatgaattgcggagtcagatccggggtcggctcaacggacaggaacagcgccaggcgttccatcatattacgcagctcgcggatgttacccggccagtcataatgcagcagcaccgtttcgctcgcctgcagaccctggcgcagtgccgcagagaacggtgcgctcagggctgccagcgagactttcaggaaagactccgccagcggtaaaatgtcggcgacacgttcacgcaacggcgggagctgcagacgcagaatgctcaggcggtagaacaggtcgcgacgaaaacggccctgttgcatatcctcttccagattgcagtgggtcgcgctaatcacgcgcacgtctaccggaaccggttgatgaccaccgacgcgggtcacttctttctcttccagcacacgcagcagacgggtttgcaatggcagcggcatctcaccgatctcgtccaggaacaaggtgccgccgtgggcaatttcaaacaaaccagcacggccaccgcgacggctacccgtgaatgcgccctcttcgtagccaaacagctcagcttccagcaggctttccgcgattgcaccgcaattaactgccacaaacggatgagatttcttaccctggcgggcatcgtgacgggcgaaatactcacgatggattgcttgcgcagccagttccttacccgtaccagtctcgccttcgatcagaacagccgcgctgctacgtgcatacagcagaatggtctggcgaacttgctccatttgagggctttggcccagcatatcacccaggacataacgggtacgcagcgcattacgcgtcgcatcgtgggtgttgtggcgcaggctcattctggtcatgtccagggcgtcgctgaacgcctgacgcaccgttgccgcgctgtagataaagatgcccgtcatgcccgcttcttcggccaagtccgtgatcagacccgcaccaaccacagcctcggtaccgttcgctttcagttcgttgatctggccacgtgcatcttcctcggtaatgtagctgcgttggtccaggcgcagattaaaggtcttttgaaacgcgaccagcgcagggatcgtttcctggtaggtgacaacgccaatcgaggaggtcagtttgcctgccttcgccagcgcctgcaagacatcgtaaccgctcggcttaatcaggatcaccggcacggacagacgggatttcaggtaggcaccattgctacccgctgcgataatggcgtcacaacgctcgttggccagctttttgcgaatgtaggtaacggctttctcgaaacccagctgaatcggagtgatgttcgccaggtgatcaaactccaggctaatgtcgcggaacaactcgaacagacgggtgacgctaacggtccaaataactggtttatcatcgttcaaacgcggtgggtgtgccatggtgaatacctcctgttaagaaaccgaatattgggtttaaacttgtttcataattgttgcaatgaaacgcggtgaaacattgcctgaaacgttaactgaaacgcatatttgcggattagttcatgactttatctctaacaaattgaaattaaacatttaattttattaaggcaattgtggcacaccccttgctttgtctttatcaacgcaaataacaagttgataacaaaagcttaggaggaaaacatagagaccggtctctctcgagtaactagttgatagagatcaagccttaacgaactaagacccccgcaccgaaaggtccgggggttttttttgaccttaaaaacataaccgaggagcagaca

SEQ ID NO: 2 - protein, 229 aa, Artificial Sequence
  Trastuzumab-Fab heavy chain A2
MEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTH

SEQ ID NO: 3 - protein, 215 aa, Artificial Sequence
  Trastuzumab-Fab light chain A2
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 228 aa, Escherichia coli
MAITISHRKQTMDDAAIQQTLAKMGIKSSDIQPAPVAGMKTVLTNSGVLYITDDGKHIIQGPMYDVSGTAPVNVTNKMLLKQLNALEKEMIVYKAPQEKHVITVFTDITCGYCHKLHEQMADYNALGITVRYLAFPRQGLDSDAEKEMKAIWCAKDKNKAFDDVMAGKSVAPASCDVDIADHYALGVQLGVSGTPAVVLSNGTLVPGYQPPKEMKEFLDEHQKMTSGK

SEQ ID NO: 5 - protein, 228 aa, Artificial Sequence
  bicistronic Trastuzumab-Fab heavy chain A3
MEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 6 - protein, 215 aa, Artificial Sequence; identical to SEQ ID NO: 3
  bicistronic Trastuzumab-Fab light chain A3

SEQ ID NO: 7 - protein, 396 aa, Artificial Sequence
  Trastuzumab-Fab heavy chain with an N-terminal amino acid sequence derived from Synechocystis sp. DnaB
MAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLASGHHHHHHGGSGSSPEIEKLSQSDIYWDSIVSITETGVEEVFDLTVPGPHNFVANDIIVHNEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 8 - protein, 383 aa, Artificial Sequence
  Trastuzumab-Fab light chain with an N-terminal amino acid sequence derived from Synechocystis sp. DnaB
MAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLASGHHHHHHGGSGSSPEIEKLSQSDIYWDSIVSITETGVEEVFDLTVPGPHNFVANDIIVHNDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 169 aa, Artificial Sequence
  N-terminal amino acid sequence derived from Synechocystis sp. DnaB that including 6xHis sequence
MAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLASGHHHHHHGGSGSSPEIEKLSQSDIYWDSIVSITETGVEEVFDLTVPGPHNFVANDIIVHN